(19) United States
(12) Patent Application Publication
(10) Pub. No.: US 2025/0101026 A1
(43) Pub. Date: Mar. 27, 2025

(54) Functionalized Allopurinol Derivatives for Treatment of Alzheimer’s Disease

(71) Applicant: BOARD OF REGENTS OF THE UNIVERSITY OF NEBRASKA, Lincoln, NE (US)

(72) Inventors: Paul Trippier, Papillion, NE (US); Ahmed Morsy, Omaha, NE (US); Krishnaiah Maddeboina, Charlotte, NC (US)

(21) Appl. No.: 18/730,842

(22) PCT Filed: Jan. 25, 2023
(86) PCT No.: PCT/US23/11500

§ 371 Date: Jul. 22, 2024

Related U.S. Application Data
(60) Provisional application No. 63/302,720, filed on Jan. 25, 2022.

(51) Int. Cl. C07D 487/04; A61K 31/519; A61K 31/5377; A61K 31/541; A61K 31/55; A61K 39/395; A61P 25/28

(52) U.S. Cl. CPC C07D 487/04; A61K 31/519; A61K 31/5377; A61K 31/541; A61K 31/55; A61K 39/3955; A61P 25/28

(57) ABSTRACT

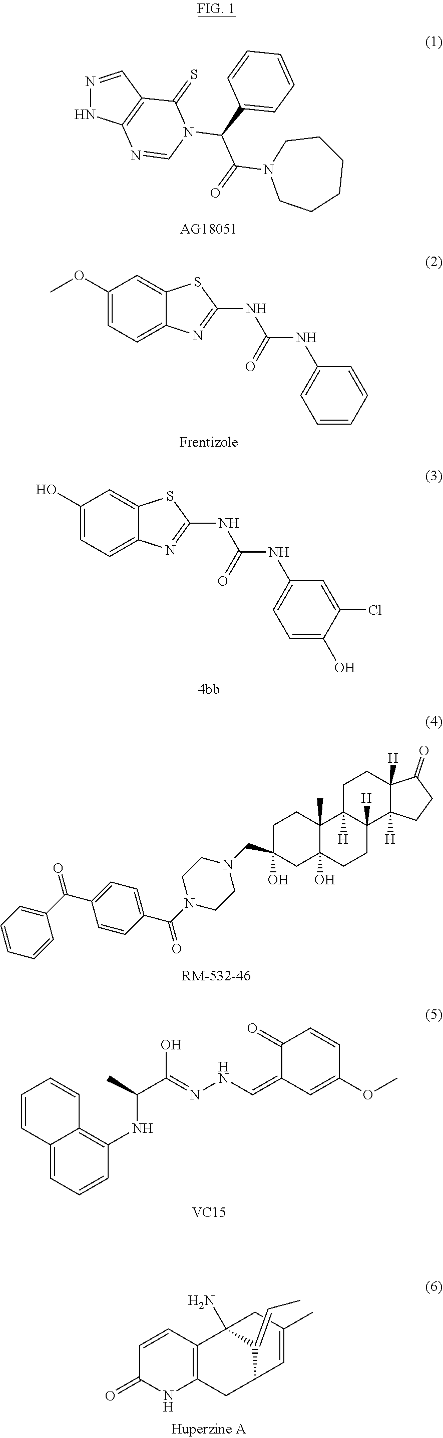

Provided herein are compounds having a structure of Formula I, Formula II, or Table 1 as disclosed herein and methods of using the disclosed compounds to inhibit a beta amyloid (Aβ)-amyloid-binding alcohol dehydrogenase (ABAD) protein-protein interaction, such as to inhibit beta amyloid (Aβ)-amyloid-binding alcohol dehydrogenase (ABAD) binding. For example, the compounds of Formula I, Formula II, and Table 1 as disclosed herein are useful for methods of treating diseases and disorders including, without limitation, Alzheimer's disease, Parkinson's disease, motor neuron disease, or spinal muscular atrophy.

### BACKGROUND

The National Institute on Aging-Alzheimer's Association guidelines for the neuropathological assessment of Alzheimer's disease (AD) define the presymptomatic phase of the disease as the stage where patients develop abnormal biomarkers denoting both amyloid-beta (Aβ) and tau deposits, with Aβ accumulation being the earliest to aggregate.¹ It is well established that these abnormal biomarker levels occur years before the onset of symptoms.² Novel treatments administered early in disease progression before the accumulation of Aβ and tau reach the threshold where neuroinflammation is triggered and irreversible neuronal damage occurs are more likely to provide effective therapy.³ There is increasing consensus that recent high profile clinical trial failures in the AD sphere may, at least in part, be due to targeting the late stage of AD when neuronal damage has already been done.⁴⁻⁷ A wider understanding of AD pathology in recent years has enabled the identification of numerous emerging targets that can improve brain physiology and allow therapeutic intervention before the pathology is too advanced and cognitive impairment to far progressed.^{4, 8, 9}

Dysfunctional brain glucose metabolism has been observed during the early stages of AD in patients; a low glucose consumption is postulated to be due to reduced glycolysis, synaptic dysfunction, and neuron loss.¹⁰ As a compensatory mechanism to this glucose hypometabolism, neurons switch to amino acids and fatty acids as alternative energy sources.¹¹ This observation suggests disruption in energetic metabolic pathways that include impaired mitochondrial function. Several reports show that mitochondrial dysfunction occurs at an early stage of AD pathology.¹²⁻¹⁴

An emerging mitochondrial target for AD is amyloid-binding alcohol dehydrogenase (ABAD), also known as type 10 17-β-hydroxysteroid dehydrogenase (17β-HSD10), L-3-hydroxyacyl-coenzyme A dehydrogenase type II (HADH2) or endoplasmic reticulum associated amyloid β-peptide-binding protein (ERAB).¹⁵ The enzyme binds to Aβ in the nanomolar range potentiating Aβ toxicity. Moreover, it has been shown to be overexpressed in the same areas of the brain most affected by AD, the cerebral cortex and hippocampus in both AD patients and transgenic amyloid precursor protein (mAPP)/ABAD mice models of AD.¹⁶ The ABAD enzyme catalyzes, with the help of a NAD⁺/NADH cofactor, the reduction and oxidation of different substrates. This includes a key role in sex steroid and neurosteroid metabolism due to 3α- and 17β-hydroxysteroid dehydrogenase activity.^{17,18} Physiological levels of the ABAD substrate estradiol in the mitochondria are a fundamental determinant of neuronal survival where they act as an antioxidant and calcium regulator.^{19, 20} Increasing evidence in the literature suggest the Aβ-ABAD protein-protein interaction (PPI) links Aβ toxicity with the mitochondrial dysfunction apparent in AD.²¹ Through its interaction with ABAD, Aβ induces a conformational change in the enzyme structure; inactivating normal enzymatic turnover. The Aβ-ABAD interaction disrupts the balance of estradiol/estrone in neurons leading to a reduction in estradiol and concomitant increase in reactive oxygen species (ROS) levels, DNA fragmentation, and apoptosis. Two other substrates of ABAD have been identified to date, peroxiredoxin-2 which functions as an antioxidant by degrading peroxides, has been shown to be elevated in AD. However, due to Cyclin-dependent kinase 5 (CDK5) elevation in the cytoplasm of neurons it has been shown to be inactivated.²² Second, endophilin-1, a member of a family of proteins that are responsible for synaptic vesicle endocytosis, mitochondrial function and receptor trafficking, its activity has been shown to be diminished in AD. Inhibition of the Aβ-ABAD PPI has been shown to offer neuroprotective effect to ameliorate Aβ-induced toxicity.²³ A decoy peptide that encompasses the region in ABAD known as the L_{D} loop, where Aβ binds and induces a conformational change, has been shown to reduce expression of peroxiredoxin II and endophilin I, both of which are elevated in AD patients and murine models of AD.¹⁶

A need exists for new strategies for treating Alzheimer's disease and related diseases and disorders, such as by inhibiting a beta amyloid (Aβ)-amyloid-binding alcohol dehydrogenase (ABAD) protein-protein interaction, e.g. inhibiting beta amyloid (Aβ)-amyloid-binding alcohol dehydrogenase (ABAD) binding.

### SUMMARY

Provided herein are compounds, or pharmaceutically acceptable salts thereof, having

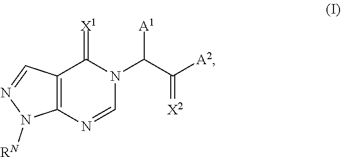

a structure of Formula I:
wherein X¹ and X² are each independently O, S, or NR^{N}; each R^{N} is independently H or C₁₋₃ alkyl; A¹ is C₃₋₁₂ cycloalkyl, 3-12 membered heterocycloalkyl having 1-4 ring heteroatoms selected from O, S, and N, C₆₋₁₀ aryl, or 5-12 membered heteroaryl having 1-4 ring heteroatoms selected from O, S, and N, wherein the cycloalkyl, heterocycloalkyl, aryl, or heteroaryl is optionally substituted with 1-3 R¹; A² is 3-12 membered heterocycloalkyl having 1-4 ring heteroatoms selected from O, S, wherein the heterocycloalkyl is optionally substituted with 1-3 R²; and each R¹ and R² is independently halogen, OH, CN, C₁₋₆ alkyl, C₁₋₆ alkyl substituted with 1-3 OH, C₁₋₆ alkyl substituted with 1-3 halogen, C₁₋₆ alkoxy, C₁₋₆ alkoxy substituted with 1-3 halogen, NO₂, NH₂, NH(C₁₋₆ alkyl), N(C₁₋₆ alkyl)₂, COOH, C(O)O—C₁₋₆ alkyl, C(O)NH₂, C(O)NH(C₁₋₆ alkyl), or C(O)N(C₁₋₆ alkyl)₂, with the proviso that the compound is not 1-(azepan-1-yl)-2-phenyl-2-(4-thioxo-1,4-dihydro-5H-pyrazolo[3,4-d]pyrimidin-5-yl)ethan-1-one. Also provided are compounds, or pharmaceutically acceptable salts thereof, having a structure listed in Table 1.

Further provided herein are pharmaceutical compositions comprising a compound or salt disclosed herein and a pharmaceutically acceptable excipient.

Also provided herein are methods of inhibiting a beta amyloid (Aβ)-amyloid-binding alcohol dehydrogenase (ABAD) protein-protein interaction in a subject in need thereof, comprising administering to the subject a therapeutically effective amount of a compound or salt disclosed herein. In some aspects, provided are methods of inhibiting beta amyloid (Aβ)-amyloid-binding alcohol dehydrogenase (ABAD) protein-protein binding comprising administering to the subject a therapeutically effective amount of a compound or salt disclosed herein.

Also provided are methods of treating or preventing a disease or disorder capable of being modulated by inhibiting beta amyloid (Aβ)-amyloid-binding alcohol dehydrogenase (ABAD) protein-protein interaction, comprising administering to a subject in need thereof a therapeutically effective amount of a compound or salt disclosed herein. In some aspects, the methods comprise treating or preventing a disease or disorder capable of being modulated by inhibiting beta amyloid (Aβ)-amyloid-binding alcohol dehydrogenase (ABAD) binding. In embodiments, the disease or disorder is Alzheimer's disease, Parkinson's disease, motor neuron disease, or spinal muscular atrophy.

### BRIEF DESCRIPTION OF THE DRAWINGS

FIG. 1 shows the structures of selected ABAD/Aβ-ABAD PPI inhibitors: AG18051 (Compound 1), Frentizole (Compound 2), 4bb (Compound 3), RM-532-46 (Compound 4), VC15 (Compound 5), and Huperzine A (Compound 6).

FIGS. 2A and 2B show the effect of Aβ and AG18051 on estradiol levels. FIG. 2A shows that estradiol levels in SH-SY5Y cells treated with Aβ alone (72 hours) and with pretreatment for 24 hours of AG18051 or HupA at the indicated concentrations. FIG. 2B shows the percentage of estradiol levels in SH-SY5Y cells treated with increasing concentration of AG18051 alone and in the presence of Aβ. n=3, ±SEM, A) One-way ANOVA; 95% Confidence Interval; *, p<0.02 **, p=0.001; ***, p=0.0007; ****, p<0.0001. B) Two-way ANOVA; 95% Confidence Interval; *, p<0.03.

FIGS. 3A and 3B show a binding model of predictive interactions between ABAD (PDB code: 1U7T) amino acid residues and (FIG. 3A) compound 14b and (FIG. 3B) compound 14h.

FIGS. 4A, 4B, 4C, and 4D show the neuroprotective effect of compounds 1 and 14b in ameliorating Aβ-induced toxicity in human SH-SY5Y ‘neuron-like’ cells. Percentage of cell viability of SH-SY5Y cells with increasing concentrations of (FIG. 4A) 1 and 14b and (FIG. 4B) Aβ. SH-SY5Y cells were pretreated with Aβ-ABAD PPI inhibitor for 24 hrs. prior to incubation with 25 μM Aβ for 48 hrs. FIG. 4C shows the percentage of cell viability measured by MTS assay. FIG. 4D shows the percentage of lactate dehydrogenase release. n=3, ±SEM, One-way ANOVA; 95% Confidence Interval; **, p<0.001; ***, p<0.0006; ****, p<0.0001.

FIGS. 5A, 5B, 5C, 5D, and 5E show that AG18051 (Compound 1) and 14b rescue Aβ-induced mitochondrial dysfunction in SH-SY5Y cells. Human SH-SY5Y ‘neuron-like’ cells were pretreated with 1 or 14b for 24 hrs. prior to incubation with 25 μM Aβ for 48 hrs. FIG. 5A shows Mitochondrial Respiration of SH-SY5Y cells, Aβ-treated SH-SY5Y cells, treated with 1 μM 1 and AB, 1 μM 14b and Aβ and 10 μM HupA and Aβ as determined by the mito stress test on an extracellular flux analyzer. FIG. 5B shows quantification of basal respiration. FIG. 5C shows proton leak. FIG. 5D shows ATP production. n=3, ±SD, one-way ANOVA; 95% confidence interval; *, p=<0.04; **, p=<0.001; ****, p=<0.0001. FIG. 5E shows maximal respiration.

FIGS. 6A, 6B, and 6C show that compound 14b rescues defective mitochondrial morphology and respiration of 5XFAD 5XFAD Alzheimer's Disease mouse model primary cortical neurons. FIG. 6A shows a representative image of NTg primary cortical neurons and 5XFAD 5XFAD primary cortical neurons cultured for 10 days and then treated with 14b at 10 and 50 μM for 48 hours and then stained with the mitochondrial outer membrane protein TOM20. FIG. 6B shows quantitation of 5XFAD 5XFAD primary cortical neuron mitochondrial apex ratio (left) and length in μm (right). n=6, ±SD, One-way ANOVA; 95% Confidence Interval; *, p=<0.05; **, p=<0.006. FIG. 6C shows the oxygen consumption rate, mitochondrial basal respiration, ATP-linked respiration and maximal respiration of NTg mouse primary cortical neurons and 5XFAD 5XFAD primary cortical neurons treated with 14b at 10 μM and 50 μM for 48 hours.

### DETAILED DESCRIPTION

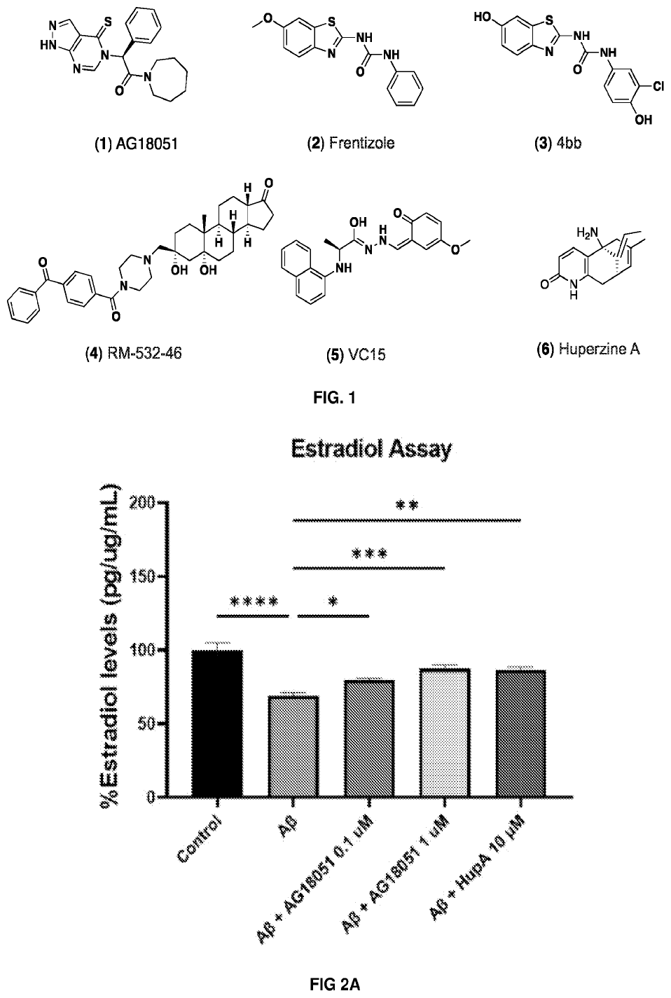

The development of small molecule inhibitors of ABAD/Aβ-ABAD PPI are in the nascent stage and can be divided into four scaffolds; fused pyrazole compounds,²³ benzothiazolyl ureas,²⁴ steroidal inhibitors,²⁵ and L_{D} loop hot spot mimetics. Examples of each of these scaffolds are shown in FIG. 1).²⁶ The fused pyrazoles were the first identified compounds and possess good potency, exemplified by AG18051 (Compound 1; IC₅₀=92 nM). However, Compound 1 suffers from poor solubility and predicted toxicity. The structure-activity relationship (SAR) of these compounds has been largely underexplored with the exception of an in silico study that predicted modifications on the phenyl and/or azepane ring would improve potency.²⁷ The benzothiazolyl ureas are based on the frentizole scaffold (Compound 2; IC₅₀=200 μM), an FDA approved immunosuppressant.²⁸ A potent compound from this class is ‘4bb’ (Compound 3; IC₅₀=1.2 μM).²⁴ However, this type of compound has been previously shown to have poor absorption, distribution, metabolism and excretion (ADME) properties and low blood-brain barrier (“BBB”) permeability,²⁹ which is a crucial consideration for a central nervous system (“CNS”) drug.³⁰ The steroidal compounds mimic the natural substrates of ABAD. A potent compound from the steroidal inhibitor class is the reversible inhibitor RM-532-46 (Compound 4). The inhibitor was tested in vitro using a HEK-293 cell line stably overexpressing ABAD and showed an IC₅₀=0.55 μM. However, when tested in an isolated enzyme-based assay the inhibitor showed an IC₅₀ of 985 μM and 710 μM using radiolabeled allopregnanolone and estradiol, respectively. Thus, direct comparison of the activity of compounds in FIG. 1 is not possible. Compound 4 has also shown activity towards type 3 17β-HSD.³¹ The L_{D} loop hotspot mimics were identified via virtual screening. The most potent inhibitor identified to date is VC15 (Compound 5; IC₅₀=4.4 μM).²⁶ In addition, the natural product alkaloid Huperzine A (Compound 6), an over the counter nutrient that is clinically approved for the treatment of AD in China,³² has recently been shown to possess ABAD inhibition activity at 10 μM concentration.³³

The compounds disclosed herein build on an expanded SAR of the AG18051 chemotype, enabled by an expedient synthetic route, that has led to the identification of more potent inhibitors that possess predicted BBB penetration greater than that of the parent and reduced toxicity. Furthermore, these novel compounds rescue Aβ-induced mitochondrial dysfunction and significantly ameliorate Aβ-induced toxicity in an SH-SY5Y cellular model and show a trend of protective effect in primary cortical neurons isolated from 5XFAD mice.

Disclosed herein are structure-activity relationship studies around the AG18051 scaffold that has previously been characterized as an Aβ-ABAD PPI inhibitor with limiting solubility and toxicity. The ABAD enzyme performs a myriad of functions that maintain healthy cellular phenotype not least of which is the regulation of estradiol levels. A growing body of evidence shows estradiol is a potent neuroprotective and neurotrophic factor that influences memory and cognition. Therefore, Aβ inhibition of its production via inhibition of ABAD, contributes to dementia which would be expected to be compounded with compounds that indiscriminately inhibit ABAD. Thus, screening for compounds that rescue Aβ-induced reduction of estradiol levels would identify Aβ-ABAD PPI inhibitors that prevent or reduce Aβ binding to ABAD.

The profile of AG18051 to serve as a hit compound for this screening campaign was confirmed by resynthesis and testing. The compound ameliorates Aβ-induced reduction of estradiol levels with an IC₅₀=1.2 μM. An expedient synthetic route beginning from commercially available allopurinol was developed that was amenable to analogue synthesis. Nineteen derivatives were synthesized that allowed assignment of a SAR and identification of several compounds with increased activity. No difference in activity was observed between amide and thioamide bioisosteres. Halogen substitution to the phenyl ring significantly reduced activity while electronegative atom functionalization of the azepane ring combined with truncation to a piperidine resulted in compounds with increased activity. Notably the electronegative atom must be extracyclic to retain activity and further truncation from piperidine to pyrrolidine to azetidine successively reduced activity. Molecular modelling predicts a hydrogen bond interaction between the hydroxyl group and Glyl99 of the ABAD protein. Derivative 14b, composed of a piperidine with 4-OH substitution (IC₅₀=0.74±11 μM) was taken forward for further evaluation.

Compound 14b was shown to be non-toxic to SH-SY5Y cells up to 100 μM and significantly rescued Aβ-induced reduction of cell viability at 1 μM in both MTS and LDH release assays. The compound rescued Aβ-mediated mitochondrial metabolic dysfunction in SH-SY5Y cells; significantly rescuing basal respiration and a showing a trend to rescue proton leak, ATP production and maximal respiration above that of AG18051 and HupA. In primary cortical neurons obtained from 5XFAD AD mouse models 14b again showed a trend to rescue dysfunctional mitochondrial metabolism. The compound significantly rescued defective mitochondrial morphology, measured by mitochondrial aspect ratio and length, in the 5XFAD neurons compared with non-AD controls.

Compound 14b and the SAR determined herein provide a blueprint for further compound design to identify more potent Aβ-ABAD PPI inhibitors that can prevent the deleterious action of Aβ binding to ABAD while preserving the protective effects of the enzyme's normal function.

Provided herein are compounds having the structure of Formula I or salts thereof, methods of inhibiting a beta amyloid (Aβ)-amyloid-binding alcohol dehydrogenase (ABAD) protein-protein interaction, e.g., inhibiting beta amyloid (Aβ)-amyloid-binding alcohol dehydrogenase (ABAD) binding, and methods of treating a disease or disorder capable of being modulated by inhibiting beta amyloid (Aβ)-amyloid-binding alcohol dehydrogenase (ABAD) protein-protein interaction in a subject, e.g., by inhibiting beta amyloid (Aβ)-amyloid-binding alcohol dehydrogenase (ABAD) binding.

The compounds disclosed herein have a structure of Formula I or a pharmaceutically acceptable salt thereof:

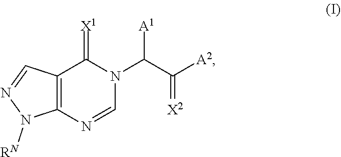

wherein
X¹ and X² are each independently O, S, or NR^{N};
each R^{N} is independently H or C₁₋₃ alkyl;
A¹ is C₃₋₁₂ cycloalkyl, 3-12 membered heterocycloalkyl having 1-4 ring heteroatoms selected from O, S, and N, C₆₋₁₀ aryl, or 5-12 membered heteroaryl having 1-4 ring heteroatoms selected from O, S, and N, wherein the cycloalkyl, heterocycloalkyl, aryl, or heteroaryl is optionally substituted with 1-3 R¹;
A² is 3-12 membered heterocycloalkyl having 1-4 ring heteroatoms selected from O, S, wherein the heterocycloalkyl is optionally substituted with 1-3 R²; and
each R¹ and R² is independently halogen, OH, CN, C₁₋₆ alkyl, C₁₋₆ alkyl substituted with 1-3 OH, C₁₋₆ alkyl substituted with 1-3 halogen, C₁₋₆ alkoxy, C₁₋₆ alkoxy substituted with 1-3 halogen, NO₂, NH₂, NH(C₁₋₆ alkyl), N(C₁₋₆ alkyl)₂, COOH, C(O)O—C₁₋₆ alkyl, C(O)NH₂, C(O)NH(C₁₋₆ alkyl), or C(O)N(C₁₋₆ alkyl)₂;
with the proviso that the compound is not 1-(azepan-1-yl)-2-phenyl-2-(4-thioxo-1,4-dihydro-5H-pyrazolo[3,4-d]pyrimidin-5-yl)ethan-1-one.

In some cases X¹ and X² are each independently O or S. In some cases at least one of X¹ and X² is O. In some cases, X¹ is O. In some cases, X¹ is S. In some cases, X² is O. In some cases, In some cases X¹ is O and X² is O. In some cases, In some cases X¹ is S and X² is O. In some cases, X¹ is NR^{N}. In some cases, X² is NR^{N}.

In some cases, at least one R^{N} is H. In some cases, each R^{N} is H. In some cases, at least one R^{N} is C₁₋₃ alkyl. In some cases, each R^{N} is C₁₋₃ alkyl. In some cases, at least one R^{N} is methyl. In some cases, each R^{N} is methyl.

In some cases, A¹ is C₃₋₁₂ cycloalkyl optionally substituted with 1-3 R¹. In some cases, A¹ is 3-12 membered heterocycloalkyl having 1-4 ring heteroatoms selected from O, S, and N and optionally substituted with 1-3 R¹. In some cases, A¹ is C₆₋₁₀ aryl optionally substituted with 1-3 R¹. In some cases, A¹ is phenyl optionally substituted with 1-3 R¹. In some cases, A¹ is 5-12 membered heteroaryl having 1-4 ring heteroatoms selected from O, S, and N and optionally substituted with 1-3 R¹. In some cases, A¹ is unsubstituted. In some cases, A¹ is substituted with 1 R¹. In some cases, A¹ is substituted with 2 R¹. In some cases, A¹ is substituted with 3 R¹.

In some cases, R¹ is halogen. In some cases, R¹ is F or Cl. In some cases, R¹ is F. In some cases, R¹ is Cl. In some cases, R¹ is OH. In some cases, R¹ is CN. In some cases, R¹ is C₁₋₆ alkyl, C₁₋₆ alkyl substituted with 1-3 OH, or C₁₋₆ alkyl substituted with 1-3 halogen. In some cases, R¹ is C₁₋₆ alkyl. In some cases, R¹ is C₁₋₆ alkyl substituted with 1-3 OH. In some cases, R¹ is C₁₋₆ alkyl substituted with 1-3 halogen. In some cases, R¹ is C₁₋₆ alkoxy or C₁₋₆ alkoxy substituted with 1-3 halogen. In some cases, R¹ is C₁₋₆ alkoxy. In some cases, R¹ is C₁₋₆ alkoxy substituted with 1-3 halogen. In some cases, R¹ NO₂. In some cases, R¹ is NH₂, NH(C₁₋₆ alkyl), or N(C₁₋₆ alkyl)₂. In some cases, R¹ is NH₂. In some cases, R¹ is NH(C₁₋₆ alkyl). In some cases, R¹ is N(C₁₋₆ alkyl)₂. In some cases, R¹ COOH, C(O)O—C₁₋₆ alkyl, C(O)NH₂, C(O)NH(C₁₋₆ alkyl), or C(O)N(C₁₋₆ alkyl)₂. In some cases, R¹ COOH. In some cases, R¹ C(O)O—C₁₋₆ alkyl. In some cases, R¹ C(O)NH₂. In some cases, R¹ C(O)NH(C₁₋₆ alkyl). In some cases, R¹ C(O)N(C₁₋₆ alkyl)₂.

In some cases, A² is 4-6 membered heterocycloalkyl having 1-4 ring heteroatoms selected from O, S, wherein the heterocycloalkyl is optionally substituted with 1-3 R². In some cases, A² is 8-12 membered heterocycloalkyl having 1-4 ring heteroatoms selected from O, S, wherein the heterocycloalkyl is optionally substituted with 1-3 R². In some cases, A² is 4-membered heterocycloalkyl having 1-4 ring heteroatoms selected from O, S. In some cases, A² is 5-membered heterocycloalkyl having 1-4 ring heteroatoms selected from O, S. In some cases, A² is 6-membered heterocycloalkyl having 1-4 ring heteroatoms selected from O, S. In some cases, A² is 8-membered heterocycloalkyl having 1-4 ring heteroatoms selected from O, S. In some cases, A² is 9-membered heterocycloalkyl having 1-4 ring heteroatoms selected from O, S. In some cases, A² is 10-membered heterocycloalkyl having 1-4 ring heteroatoms selected from O, S. In some cases, A² is 11-membered heterocycloalkyl having 1-4 ring heteroatoms selected from O, S. In some cases, A² is 12-membered heterocycloalkyl having 1-4 ring heteroatoms selected from O, S. In some cases, A² is 3-12 membered heterocycloalkyl having at least 1 ring N heteroatom. In some cases, A² is 4-6 membered heterocycloalkyl having at least 1 ring N heteroatom. In some cases, A² is 8-12 membered heterocycloalkyl having at least 1 ring N heteroatom. In some cases, A² is 4-membered heterocycloalkyl having at least 1 ring N heteroatom. In some cases, A² is 5-membered heterocycloalkyl having at least 1 ring N heteroatom. In some cases, A² is 6-membered heterocycloalkyl having at least 1 ring N heteroatom. In some cases, A² is 8-membered heterocycloalkyl having at least 1 ring N heteroatom. In some cases, A² is 9-membered heterocycloalkyl having at least 1 ring N heteroatom. In some cases, A² is 10-membered heterocycloalkyl having at least 1 ring N heteroatom. In some cases, A² is 11-membered heterocycloalkyl having at least 1 ring N heteroatom. In some cases, A² is 12-membered heterocycloalkyl having at least 1 ring N heteroatom. In some cases, A² is morpholino or thiomorpholino. In some cases, A² is morpholino. In some cases, A² is thiomorpholino.

In some cases, the compound is a compound of Formula (II):

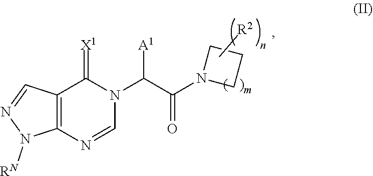

wherein m is an integer from 1 to 3; and n is an integer from 0 to 2. In some cases, m is 1. In some cases, m is 2. In some cases, m is 3. In some cases, n is 0. In some cases, n is 1 or 2. In some cases, n is 1. In some cases, n is 2.

In some cases, R² is halogen. In some cases, R² is OH or C₁₋₆ alkoxy. In some cases, R² is OH. In some cases, R² is CN. In some cases, R² is C₁₋₆ alkyl, C₁₋₆ alkyl substituted with 1-3 OH, or C₁₋₆ alkyl substituted with 1-3 halogen. In some cases, R² is C₁₋₆ alkyl. In some cases, R² is C₁₋₆ alkyl substituted with 1-3 OH. In some cases, R² is C₁₋₆ alkyl substituted with 1-3 halogen. In some cases, R² is C₁₋₆ alkoxy or C₁₋₆ alkoxy substituted with 1-3 halogen. In some cases, R² is C₁₋₆ alkoxy. In some cases, R² is OCH₃. In some cases, R² is C₁₋₆ alkoxy substituted with 1-3 halogen. In some cases, R² NO₂. In some cases, R² is NH₂, NH(C₁₋₆ alkyl), or N(C₁₋₆ alkyl)₂. In some cases, R² is NH₂. In some cases, R² is NH(C₁₋₆ alkyl). In some cases, R² is N(C₁₋₆ alkyl)₂. In some cases, R² COOH, C(O)O—C₁₋₆ alkyl, C(O)NH₂, C(O)NH(C₁₋₆ alkyl), or C(O)N(C₁₋₆ alkyl)₂. In some cases, R¹ COOH. In some cases, R² C(O)O—C₁₋₆ alkyl. In some cases, R² C(O)NH₂. In some cases, R² C(O)NH(C₁₋₆ alkyl). In some cases, R² C(O)N(C₁₋₆ alkyl)₂.

As used herein, the term “alkyl” refers to straight chained and branched saturated hydrocarbon groups containing one to six carbon atoms. The term Cₙ means the alkyl group has “n” carbon atoms. For example, C₄ alkyl refers to an alkyl group that has 4 carbon atoms. C₁₋₆ alkyl refers to an alkyl group having a number of carbon atoms encompassing the entire range (e.g., 1 to 6 carbon atoms), as well as all subgroups (e.g., 1-6, 2-6, 3-6, 4-6, 5-6, 1-5, 2-5, 3-5, 4-5, 1-4, 2-4, 3-4, 1-3, 2-3, 1-2, 1, 2, 3, 4, 5, and 6 carbon atoms), and C₁₋₃ alkyl refers to an alkyl group having a number of carbon atoms encompassing the entire range (e.g., 1 to 3 carbon atoms), as well as all subgroups (e.g., 1-3, 1-2, 2-3, 1, 2, and 3 carbon atoms). Nonlimiting examples of alkyl groups include, methyl, ethyl, n-propyl, isopropyl, n-butyl, sec-butyl (2-methylpropyl), t-butyl (1,1-dimethylethyl), and hexyl. Unless otherwise indicated, an alkyl group can be an unsubstituted alkyl group or a substituted alkyl group.

The term “alkoxy” used herein refers to an —O-alkyl group.

As used herein, the term “halogen means F, Cl, Br, or I.

The term “cycloalkyl” refers to a non-aromatic monocyclic, fused, bridged or spiro ring system whose ring atoms are carbon and which can be saturated or have one or more units of unsaturation. The carbocycle can have three to twelve ring carbon atoms. In some embodiments, the number of carbon atoms is 5 to 6. In some embodiments, the number of carbon atoms is 6. “Fused” bicyclic ring systems comprise two rings which share two adjoining ring atoms. Bridged bicyclic group comprise two rings which share three or four adjacent ring atoms. Spiro bicyclic ring systems share one ring atom. Cycloalkyl groups can include cycloalkenyl groups. Specific examples include, but are not limited to, cyclohexyl, cyclopentyl, cyclopropyl, and cyclobutyl. Unless otherwise indicated, a cycloalkyl group can be unsubstituted or substituted with one or more, and in particular one to three groups as described herein.

The term “heterocycloalkyl” as used herein refers to a non-aromatic monocyclic, fused, spiro or bridged ring system which can be saturated or contain one or more units of unsaturation, having three to twelve ring atoms in which one or more (e.g., one to four, or one, two, three, or four) ring atoms is a heteroatom selected from, N, S, and O. An “N-heterocycloalkyl” indicates that at least one of the ring heteroatoms is a nitrogen atom. In some embodiments, the heterocycloalkyl comprises 4-6 ring members. In some embodiments, the heterocycloalkyl comprises 4 ring members. In some embodiments, the heterocycloalkyl comprises 5 ring members. In some embodiments, the heterocycloalkyl comprises 6 ring members. In some embodiments, the heterocycloalkyl is piperidinyl. Non-limiting examples of heterocycloalkyls include, but are not limited to, quinuclidinyl, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrothiophenyl, morpholino (including, for example, 3-morpholino, 4-morpholino), 2-thiomorpholino, 3-thiomorpholino, 4-thiomorpholino, 1-pyrrolidinyl, 2-pyrrolidinyl, 3-pyrrolidinyl, pyrrolidin-2-one, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)-benzimidazol-2-onyl, and 1,3-dihydro-imidazol-2-onyl. Unless otherwise indicated, a heterocycloalkyl group can be unsubstituted or substituted with one or more, and in particular one to three groups as described herein.

As used herein, the term “aryl” refers to a cyclic aromatic group, such as a monocyclic aromatic group, e.g., phenyl. Unless otherwise indicated, an aryl group can be unsubstituted or substituted with one or more, and in particular one to three groups as described herein. A C₆₋₁₀ aryl group is an aryl group that has 6-10 ring carbon atoms. Aryl groups can be isolated (e.g., phenyl) or fused to another aryl group (e.g., naphthyl, anthracenyl). Exemplary aryl groups include, but are not limited to, phenyl, naphthyl, and the like.

As used herein, the term “heteroaryl” refers to a cyclic aromatic ring having five to twelve total ring atoms (e.g., a monocyclic aromatic ring with 5-12 total ring atoms), and containing one to four heteroatoms selected from nitrogen, oxygen, and sulfur atoms in the aromatic ring. Unless otherwise indicated, a heteroaryl group can be unsubstituted or substituted with one or more, and in particular one to three, substituents as described herein. In some cases, the heteroaryl group is substituted with one or more alkyl groups, such as methyl groups. Examples of heteroaryl groups include, but are not limited to, thienyl, furyl, pyridyl, pyrrolyl, pyrazolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

A used herein, the term “substituted,” when used to modify a chemical functional group, unless noted otherwise, refers to the replacement of at least one hydrogen radical on the functional group with a substituent. Substituents can include, but are not limited to, alkyl, cycloalkyl, alkynyl, heterocycloalkyl, thioether, polythioether, aryl, heteroaryl, hydroxyl, oxy, alkoxy, heteroalkoxy, aryloxy, heteroaryloxy, ester, thioester, carboxy, cyano, nitro, amino, amido, acetamide, and halogen (e.g., fluoro, chloro, bromo, or iodo). When a chemical functional group includes more than one substituent, the substituents can be bound to the same carbon atom or to two or more different carbon atoms.

The salts, e.g., pharmaceutically acceptable salts, of compounds of Formula (I), Formula (II), or Table 1 may be prepared by reacting the appropriate base or acid with a stoichiometric equivalent of compound of Formula (I), Formula (II), or Table 1.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include anions, for example sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, and mandelate. In one embodiment, pharmaceutically acceptable acid addition salts include those formed with mineral acids such as hydrochloric acid and hydrobromic acid, and especially those formed with organic acids such as maleic acid.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′-dibenzylethylenediamine, chloroprocaine, choline, diethanolamine, dicyclohexylamine, ethylenediamine, N-methylglucamine, and procaine.

Specifically contemplated compounds of the disclosed Formula (I) include the compounds having a structure shown in Table 1 below.

TABLE 1

| Compound No. | Structure |
|---|---|
| 13a | 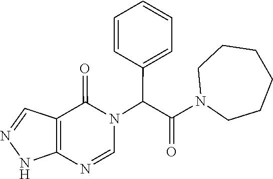 |
| 13b | 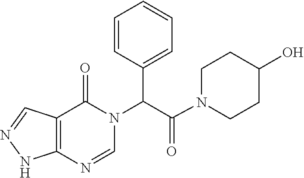 [SMILES: O=C(C(c1ccccc1)n1cnc2[nH]ncc2c1=O)N1CCC(O)CC1] |
| 14b | 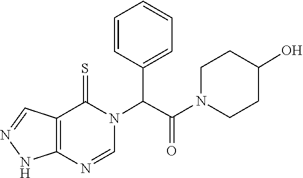 [SMILES: O=C(C(c1ccccc1)n1cnc2[nH]ncc2c1=S)N1CCC(O)CC1] |
| 13c | 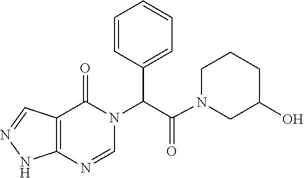 [SMILES: O=C(C(c1ccccc1)n1cnc2[nH]ncc2c1=O)N1CCCC(O)C1] |
| 14c | 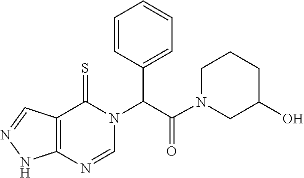 [SMILES: O=C(C(c1ccccc1)n1cnc2[nH]ncc2c1=S)N1CCCC(O)C1] |
| 13d | 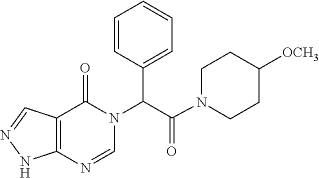 [SMILES: COC1CCN(C(=O)C(c2ccccc2)n2cnc3[nH]ncc3c2=O)CC1] |
| 14d | 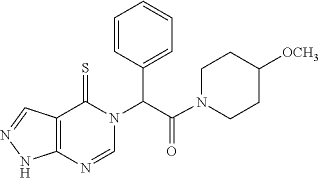 [SMILES: COC1CCN(C(=O)C(c2ccccc2)n2cnc3[nH]ncc3c2=S)CC1] |
| 13e | 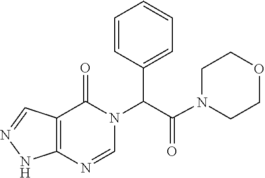 [SMILES: O=C(C(c1ccccc1)n1cnc2[nH]ncc2c1=O)N1CCOCC1] |
| 14e | 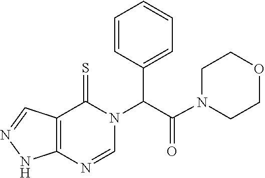 [SMILES: O=C(C(c1ccccc1)n1cnc2[nH]ncc2c1=S)N1CCOCC1] |
| 13f | 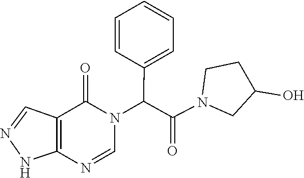 |
| 13g | 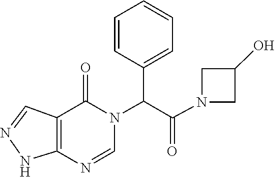 |
| 14g | 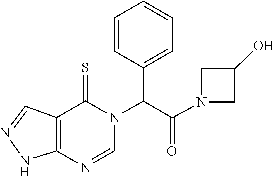 |
| 13h | 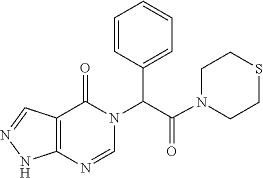 [SMILES: O=C(C(c1ccccc1)n1cnc2[nH]ncc2c1=O)N1CCSCC1] |
| 14h | 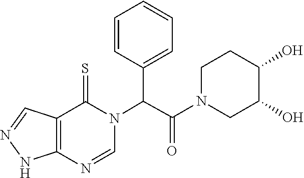 [SMILES: O=C(C(c1ccccc1)n1cnc2[nH]ncc2c1=S)N1CC[C@H](O)[C@H](O)C1] |
| 14h bis | 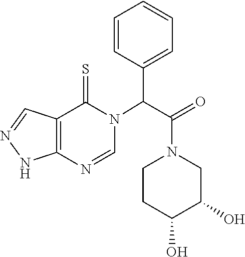 [SMILES: O=C(C(c1ccccc1)n1cnc2[nH]ncc2c1=S)N1CC[C@@H](O)[C@@H](O)C1] |
| 13i | 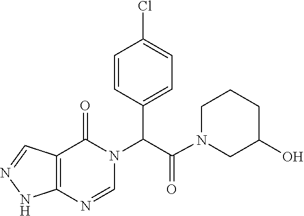 [SMILES: O=C(C(c1ccc(Cl)cc1)n1cnc2[nH]ncc2c1=O)N1CCCC(O)C1] |
| 13j | 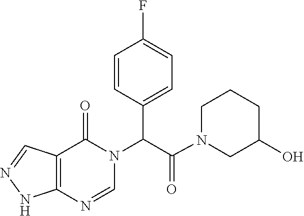 [SMILES: O=C(C(c1ccc(F)cc1)n1cnc2[nH]ncc2c1=O)N1CCCC(O)C1] |
| 13k | 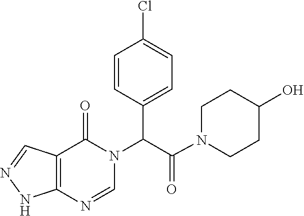 [SMILES: O=C(C(c1ccc(Cl)cc1)n1cnc2[nH]ncc2c1=O)N1CCC(O)CC1] |
| 13l | 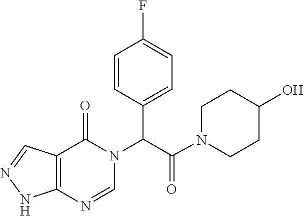 [SMILES: O=C(C(c1ccc(F)cc1)n1cnc2[nH]ncc2c1=O)N1CCC(O)CC1] |

In some cases, the compound is compound 14b or 14h or a pharmaceutically acceptable salt thereof:

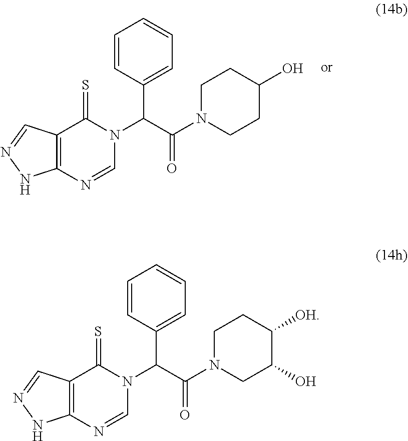 [SMILES: O=C(C(c1ccccc1)n1cnc2[nH]ncc2c1=S)N1CCC(O)CC1 ; "or" ; O=C(C(c1ccccc1)n1cnc2[nH]ncc2c1=S)N1CC[C@H](O)[C@H](O)C1]

### Therapeutic Methods

Provided herein are compound capable of inhibiting a beta amyloid (Aβ)-amyloid-binding alcohol dehydrogenase (ABAD) protein-protein interaction, e.g., inhibiting beta amyloid (Aβ)-amyloid-binding alcohol dehydrogenase (ABAD) binding, as exemplified by compounds of Formula (I), Formula (II), and Table 1, for the treatment of a variety of diseases and conditions wherein inhibition of a beta amyloid (Aβ)-amyloid-binding alcohol dehydrogenase (ABAD) protein-protein interaction, e.g., inhibition of beta amyloid (Aβ)-amyloid-binding alcohol dehydrogenase (ABAD) binding, has a beneficial effect. In one embodiment, provided is a method of inhibiting a beta amyloid (Aβ)-amyloid-binding alcohol dehydrogenase (ABAD) protein-protein interaction, e.g., inhibiting beta amyloid (Aβ)-amyloid-binding alcohol dehydrogenase (ABAD) binding in cells comprising contacting the cell with the compound or salt of Formula (I), Formula (II), or Table 1 in an amount effective to inhibit a beta amyloid (Aβ)-amyloid-binding alcohol dehydrogenase (ABAD) protein-protein interaction, e.g., to inhibit beta amyloid (Aβ)-amyloid-binding alcohol dehydrogenase (ABAD) binding. In a further embodiment, the method decreases a beta amyloid (Aβ)-amyloid-binding alcohol dehydrogenase (ABAD) protein-protein interaction, e.g., decreases beta amyloid (Aβ)-amyloid-binding alcohol dehydrogenase (ABAD) binding. In some cases, provided herein are methods of treating or preventing a disease or disorder capable of being modulated by inhibiting beta amyloid (Aβ)-amyloid-binding alcohol dehydrogenase (ABAD) protein-protein interaction, e.g., capable of being modulated by inhibiting beta amyloid (Aβ)-amyloid-binding alcohol dehydrogenase (ABAD) binding in a subject comprising administering to the subject a therapeutically effective amount of a compound of Formula (I), Formula (II), or Table 1. In some cases, the disorder is Alzheimer's disease, Parkinson's disease, motor neuron disease, or spinal muscular atrophy.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound of Formula (I), Formula (II), or Table 1 to an individual in need of such treatment.

The term “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The compounds described herein therefore can be used to treat a variety of diseases and conditions where modulation (e.g., inhibition or activation) of a beta amyloid (Aβ)-amyloid-binding alcohol dehydrogenase (ABAD) protein-protein interaction, e.g., modulating beta amyloid (Aβ)-amyloid-binding alcohol dehydrogenase (ABAD) binding provides a benefit. In some embodiments, inhibiting a beta amyloid (Aβ)-amyloid-binding alcohol dehydrogenase (ABAD) protein-protein interaction, e.g., inhibiting beta amyloid (Aβ)-amyloid-binding alcohol dehydrogenase (ABAD) binding, provides a benefit. Examples of such diseases and conditions include, but are not limited to Alzheimer's disease, Parkinson's disease, motor neuron disease, or spinal muscular atrophy.

Presented herein are methods of administering a compound or salt thereof as disclosed herein (e.g., a compound of Formula (I), Formula (II), or Table 1) as the neat compound or as a pharmaceutical composition orally, intravenously, or parenterally. In some cases, the compound of Formula (I), Formula (II), or Table 1 or salt thereof is administered orally. Administration of a pharmaceutical composition, or neat compound of Formula (I), Formula (II), or Table 1, can be performed during or after the onset of the disease or condition of interest. Typically, the pharmaceutical compositions are sterile, and contain no toxic, carcinogenic, or mutagenic compounds that would cause an adverse reaction when administered. Further provided are kits comprising a compound of Formula (I), Formula (II), or

Table 1 and, optionally, a second therapeutic agent useful in the treatment of diseases and conditions wherein where inhibiting a beta amyloid (Aβ)-amyloid-binding alcohol dehydrogenase (ABAD) protein-protein interaction, e.g., inhibiting beta amyloid (Aβ)-amyloid-binding alcohol dehydrogenase (ABAD) binding provides a benefit, packaged separately or together, and an insert having instructions for using these active agents. The methods disclosed herein can further comprise administering one or more additional therapeutics to the subject. In cases where the compound disclosed herein, e.g., a compound of Formula (I), Formula (II), or Table 1 or a salt thereof, or a pharmaceutical composition thereof, is for use in a method of treating a disease or disorder disclosed herein, the compound or composition for use can be further formulated for use with one or more additional therapeutics. In cases where the compound disclosed herein, e.g., a compound of Formula (I), Formula (II), or Table 1 or a salt thereof, or a pharmaceutical composition thereof, is for use in the manufacture of the medicament for treating a disease or disorder disclosed herein, the medicament can further comprise one or more additional therapeutics. In some cases, the one or more additional therapeutics comprise Aducanumab. In some cases, the one or more additional therapeutics is Aducanumab.

### Dosing and Pharmaceutical Formulations

The term “therapeutically effective amount,” as used herein, refers to an amount of a compound sufficient to treat, ameliorate, or prevent the identified disease or condition, or to exhibit a detectable therapeutic, prophylactic, or inhibitory effect. The effect can be detected by, for example, an improvement in clinical condition, reduction in symptoms, or by any of the assays or clinical diagnostic tests described herein or known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

Dosages of the therapeutic can alternately be administered as a dose measured in mg/kg. Contemplated mg/kg doses of the disclosed therapeutics include about 0.001 mg/kg to about 1000 mg/kg. Specific ranges of doses in mg/kg include about 0.1 mg/kg to about 500 mg/kg, about 0.5 mg/kg to about 200 mg/kg, about 1 mg/kg to about 100 mg/kg, about 1 mg/kg to about 50 mg/kg, about 1 mg/kg to about 40 mg/kg, and about 5 mg/kg to about 30 mg/kg.

A compound of Formula (I), Formula (II), or Table 1 used in a method described herein can be administered in an amount of about 0.005 to about 750 milligrams per dose, about 0.05 to about 500 milligrams per dose, or about 0.5 to about 250 milligrams per dose. For example, a compound of Formula (I), Formula (II), or Table 1 can be administered, per dose, in an amount of about 0.005, 0.05, 0.5, 1, 2, 3, 4, 5, 10, 15, 20, 30, 40, 50, 100, 150, 200, 250, 300, 350, 400, 450, 500, 550, 600, 650, 700, or 750 milligrams, including all doses between 0.005 and 750 milligrams.

As herein, the compounds described herein may be formulated in pharmaceutical compositions with a pharmaceutically acceptable excipient, carrier, or diluent. The compound or composition comprising the compound is administered by any route that permits treatment of the disease or condition. One route of administration is oral administration. Additionally, the compound or composition comprising the compound may be delivered to a patient using any standard route of administration, including parenterally, such as intravenously, intraperitoneally, intrapulmonary, subcutaneously or intramuscularly, intrathecally, topically, transdermally, rectally, orally, nasally or by inhalation. Slow release formulations may also be prepared from the agents described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or a compound as disclosed herein can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

In an embodiment, the pharmaceutical compositions are formulated with one or more pharmaceutically acceptable excipient, such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, the pH is adjusted to a range from about pH 5.0 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein, or may include a second active ingredient useful in the treatment or prevention of a disorder as disclosed herein (e.g., an anticancer agent or an anti-inflammatory agent).

Formulations, e.g., for parenteral or oral administration, are most typically solids, liquid solutions, emulsions or suspensions, while inhalable formulations for pulmonary administration are generally liquids or powders. A pharmaceutical composition can also be formulated as a lyophilized solid that is reconstituted with a physiologically compatible solvent prior to administration. Alternative pharmaceutical compositions may be formulated as syrups, creams, ointments, tablets, and the like.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The pharmaceutical compositions described herein are formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules wherein the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents (e.g., sodium carboxymethylcellulose, methylcellulose, hydroxypropyl methylcellulose, sodium alginate, polyvinylpyrrolidone, gum tragacanth, gum acacia); dispersing or wetting agents (e.g., a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate)); and thickening agents (e.g., carbomer, beeswax, hard paraffin or cetyl alcohol). The suspensions may also contain one or more preservatives (e.g., acetic acid, methyl or n-propyl p-hydroxy-benzoate); one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In one embodiment, the active compound is dissolved in DMSO and diluted with water.

The pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In some embodiments, the compounds described herein may be formulated for oral administration in a lipid-based formulation suitable for low solubility compounds. Lipid-based formulations can generally enhance the oral bioavailability of such compounds.

As such, pharmaceutical compositions comprise a therapeutically or prophylactically effective amount of a compound described herein, together with at least one pharmaceutically acceptable excipient selected from the group consisting of medium chain fatty acids and propylene glycol esters thereof (e.g., propylene glycol esters of edible fatty acids, such as caprylic and capric fatty acids) and pharmaceutically acceptable surfactants, such as polyoxyl 40 hydrogenated castor oil.

In some embodiments, cyclodextrins may be added as aqueous solubility enhancers. Exemplary cyclodextrins include hydroxypropyl, hydroxyethyl, glucosyl, maltosyl and maltotriosyl derivatives of α-, β-, and γ-cyclodextrin. A specific cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound described herein.

### General Synthesis of Compounds

The compounds disclosed herein can be synthesized through any means available to the synthetic chemist and in view of the guidance of the schemes below. Non-limiting examples for preparing compounds disclosed herein are provided below.

An expedient synthetic route to rapidly access a wide variety of structurally diverse derivatives of AG18051 was developed (Scheme 1). The originally reported synthetic route,³⁴ proved highly challenging, resulting in low yields of both target product and intermediates. Advantageous features of the syntheses disclosed herein include rapid access to the amide bioisostere motif of AG18051 in just four steps and high overall yield compared with the original eight step route. The addition of one step allows access to the original thioamide motif. It is notable that lower-yielding steps that drive down overall yield occur early in the synthesis and provide almost quantitative recovery of starting material.

Commercially available allopurinol (7) undergoes chemoselective protection with di-tert-butyl dicarbonate to yield 8, with its structure unequivocally assigned by HMBC ¹H-¹⁴N NMR (SI Figure S1). Without wishing to be bound by theory, exposure of carbonate 8 to sodium hydride results in abstraction of a proton and nucleophilic attack of a suitably substituted methyl α-bromophenylacetate to afford methyl ester 9 with concomitant deprotection of the Boc carbamate in almost quantitative yield. At this point the synthetic route branches to afford access to either amide or thioamide bioisoteres of the hit compound. Without wishing to be bound by theory, exposure of amide (9) to Lawesson's reagent affords thioamide 10. Without wishing to be bound by theory, subsequent hydrolysis of 9 or 10 affords carboxylic acid 11 and 12 respectively, and coupling of a suitably substituted nitrogen heterocycle employing the coupling agent BOP, afforded final compounds of types 13 and 14.

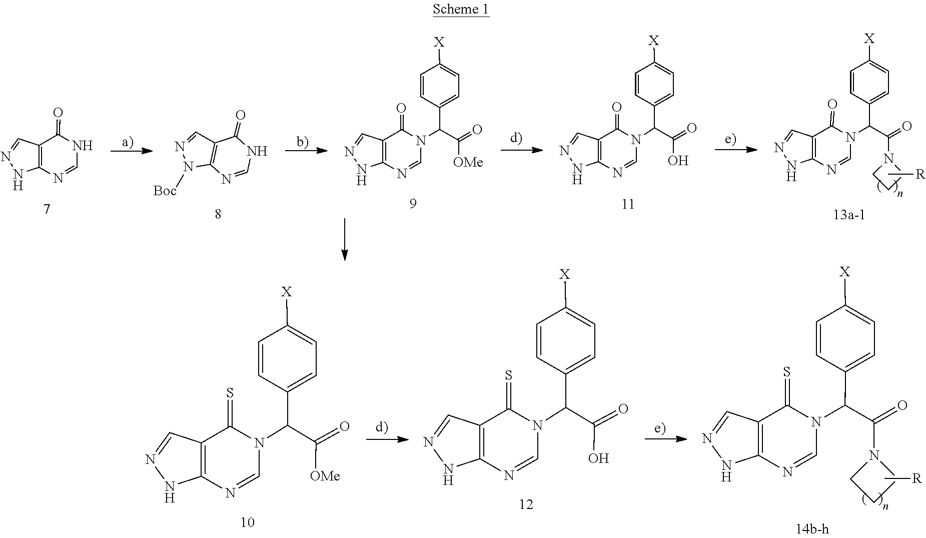

Reagents and conditions: (a) DIPEA, DMAP, (Boc)₂O, THF; (b) NaH, LiBr, DMF, suitably substituted methyl α-bromophenylacetate; (c) Lawesson's Reagent, PhMe; (d) NaOH, THF; (e) BOP, DIPEA, DMF, appropriately substituted amine.

Further examples are provided below. They should, however, not be construed as limiting the scope. All citations throughout the disclosure are hereby expressly incorporated by reference.

### EXAMPLES

### Preparation of Compounds

### General Synthetic Methods

All reactions were carried out in oven- or flame-dried glassware under nitrogen atmosphere unless otherwise noted. Reaction progress was monitored by thin-layer chromatography carried out on silica gel plates (2.5 cm×7.5 cm, 200 μm thick, 60 F254) and visualized using UV (254 nm) or by potassium permanganate and/or phosphomolybdic acid solution and/or ninhydrin as an indicator. Flash column chromatography was performed with silica gel (40-63 μm, 60 Å) using the mobile phase indicated in protocols. Solvents and reagents were purchased from commercial sources and were used without further purification, except as indicated.

¹H and ¹³C NMR spectra were recorded on a 500 MHz or 400 MHZ spectrometer. The chemical shifts of ¹H NMR are reported in parts per million (ppm) relative to internal standard tetramethylsilane or residual solvent peak. ¹³C NMR chemical shifts are reported in ppm with the solvents (CDCl₃: 77.23 ppm, CD₃OD: 49.15 ppm, DMSO-d₆: 39.51 ppm). Multiplicities are indicated by s (single), d (doublet), dd (doublet of doublets), t (triplet), q (quartet), m (multiplet), and br (broad). Chemical shifts (δ) are reported in parts per million (ppm) and coupling constants (J) are reported in hertz. High resolution mass spectra (HRMS) were recorded were acquired in positive ion mode with an LC/TOF spectrometer using an ESI source coupled to an LC system running in reverse phase with a C18 (80 Å, 2.1×50 mm, 1.8 μm) column using solvent A (water with 0.1% Formic acid), solvent B (acetonitrile with 0.1% formic acid), and a flow rate of 0.6 mL/min starting a mixture of 95% A and 5% B. Solvent B was gradually increased to 95% at 5 min, held at 95% until 6 min, then gradually ramped back down to 5% at 8.0 min. HPLC purity data for all final compounds were performed on an ultra performance liquid chromatography (UPLC) system with TUV (254 nm) detector using a C18 5μ column (4.6×150 mm) using solvent A (water with 0.1% Trifluoroacetic acid), solvent B (methanol with 0.1% Trifluoroacetic acid), and a flow rate of 0.8 mL/min starting a mixture of 90% A and 10% B. Solvent B was gradually increased to 90% for 20 min. All compounds were evaluated to be consistent with their HRMS data.

### Synthesis of Intermediates

Tert-butyl 4-oxo-4,5-dihydro-1H-pyrazolo[3,4-d]pyrimidine-1-carboxylate (8). To a suspension of 1,5-dihydro-4H-pyrazolo[3,4-d]pyrimidin-4-one (200 mg, 1.47 mmol,) in THF (10 mL) was added N,N-diisopropylethylamine (0.75 mL, 4.28 mmol), 4-dimethylaminopyridine (17.5 mg, 0.14 mmol) and di-tert-butyl dicarbonate (641 mg, 2.94 mmol). The reaction was stirred at refluxed overnight. The reaction mixture was allowed to cool to room temperature, and water (30 mL) was added slowly with stirring resulting in a solid. The mixture was extracted with ethyl acetate (3×20 mL), the organic layer was separated and dried (Na₂SO₄), filtered and evaporated in vacuo. Purification by column chromatography using DCM/MeOH gradient (20:1, 10:1) provided the title compound. R_{f}: 0.42 (DCM/MeOH, 10:1). ¹H NMR (400 MHZ, CDCl₃-d) δ ppm 1.65 (s, 9H), 8.23, (s, 1H), 8.44 (s, 1H). ¹³C NMR (100 MHZ, CDCl₃-d) δ_{C}: 27.97, 76.73, 76.73, 87.04, 108.97, 138.05, 147.14, 155.25.

Methyl-2-(4-oxo-1,4-dihydro-5H-pyrazolo[3,4-d]pyrimidin-5-yl)-2-phenylacetate (9). To a solution of tert-butyl 4-oxo-4,5-dihydro-1H-pyrazolo[3,4-d]pyrimidine-1-carboxylate (8) (200 mg, 0.85 mmol) in DMF (4 mL) was added sodium hydride (42 mg, 1.02 mmol) and lithium bromide (149 mg, 1.7 mmol). The solution was stirred for 30 minutes at room temperature. Methyl α-bromophenylacetate (136 μL, 0.85 mmol) was added and the reaction was stirred at room temperature for 4 hours. Water was added to the reaction mixture and was extracted with ethyl acetate (3×20 mL). The organic layer was separated and dried (Na₂SO₄), filtered and evaporated in vacuo. Purification by column chromatography using hexane/EtOAc gradient (3:1, 2:1, 1:1) provided the title compound. R_{f}: 0.18 (hexane/EtOAc, 1:1).¹H NMR (400 MHZ, CDCl₃-d) δ ppm 3.86 (s, 3H), 6.83 (s, 1H), 7.33-7.38 (m, 2H), 7.41-7.48 (m, 3H), 7.92 (s, 1 H), 8.22 (s, 1H). ¹³C NMR (100 MHZ, CDCl₃-d) δ_{C}: 30.32, 35.62, 52.31, 59.85, 128.97, 129.26, 129.30, 133.21, 148.26, 163.50, 169.50.

Methyl-2-phenyl-2-(4-thioxo-1,4-dihydro-5H-pyrazolo[3,4-d]pyrimidin-5-yl)acetate (10). To a solution of methyl(S)-2-(4-oxo-1,4-dihydro-5H-pyrazolo[3,4-d]pyrimidin-5-yl)-2-phenylacetate (9) (300 mg, 1.05 mmol) in toluene (10 mL) was added Lawesson's Reagent (848.4 mg, 2.10 mmol). The reaction mixture was stirred and refluxed for 2 hours. The reaction mixture was allowed to cool to room temperature, and water (30 mL) was added slowly with stirring to provide a solid. The mixture was extracted with ethyl acetate (3×20 mL), the organic layer was separated and dried (Na₂SO₄), filtered, and evaporated in vacuo. Purification by column chromatography using DCM/MeOH gradient (20:1, 10:1) provided the title compound. R_{f}: 0.32 (DCM/MeOH, 10:1). ¹H NMR (400 MHZ, CDCl₃-d) δ ppm 3.86 (s, 3H), 7.33-7.38 (m, 2H), 7.43-7.47 (m, 3H), 7.99-8.02 (m, 1H), 8.07 (s, 1H), 8.35 (s, 1H). ¹³C NMR (100 MHZ, CDCl₃-d) δ_{C}: 53.44, 63.43, 117.70, 128.95, 129.89, 133.51, 139.06, 146.28, 148.34, 169.17, 181.25.

2-(4-oxo-1,4-dihydro-5H-pyrazolo[3,4-d]pyrimidin-5-yl)-2-phenylacetic acid (11). To a solution of methyl(S)-2-(4-oxo-1,4-dihydro-5H-pyrazolo[3,4-d]pyrimidin-5-yl)-2-phenylacetate (9) (240 mg, 0.85 mmol) in THF (5 mL) was added of sodium hydroxide (0.25N, 6.75 mL, 1.69 mmol). The reaction mixture was stirred at room temperature for 2 hours and then quenched using of hydrochloric acid (0.25N, 6.75 mL, 1.69 mmol). The solution was air-dried at room temperature to afford the title compound. ¹H NMR (400 MHZ, MeOD-d₄) δ ppm 6.65 (s, 1H), 7.26-7.37 (m, 5H), 7.73 (s, 1H), 8.07 (s, 1H). ¹³C NMR (100 MHZ, MeOD-d₄) δ_{C}: 60.82, 127.76, 128.57, 128.97, 138.33, 145.72, 174.00.

2-phenyl-2-(4-thioxo-1,4-dihydro-5H-pyrazolo[3,4-d]pyrimidin-5-yl) acetic acid (12). To a solution of methyl(S)-2-phenyl-2-(4-thioxo-1,4-dihydro-5H-pyrazolo[3,4-d]pyrimidin-5-yl)acetate (10) (240 mg, 0.8 mmol) in THF (5 mL) was added of sodium hydroxide (0.25N, 6.75 mL, 1.69 mmol). The reaction mixture was stirred at room temperature for 2 hours and then quenched using of hydrochloric acid (0.25N, 6.75 mL, 1.69 mmol). The solution was air-dried at room temperature to afford the title compound. ¹H NMR (400 MHZ, CDCl₃-d) δ ppm 7.33-7.38 (m, 2H), 7.43-7.47 (m, 3H), 7.99-8.02 (m, 1H), 8.07 (s, 1H), 8.35 (s, 1H). ¹³C NMR (100 MHz, CDCl₃-d) δ_{C}: 67.43, 119.70, 128.85, 129.87, 133.41, 139.76, 146.18, 148.74, 169.87, 181.35.

General Synthetic Procedure for Amide Bioisostere Derivatives. To a solution of(S)-2-(4-oxo-1,4-dihydro-5H-pyrazolo[3,4-d]pyrimidin-5-yl)-2-phenylacetic acid (13) (200 mg, 0.74 mmol) in DMF (5 mL) were added (Benzotriazol-1-yloxy)tris(dimethylamino)phosphonium hexafluorophosphate (392 mg, 0.88 mmol), N,N-diisopropylethylamine (260 μL, 1.48 mmol) and the respective substituted amine (0.74 mmol). The reaction mixture was stirred at room temperature overnight. Water was added to the reaction mixture and was extracted with ethyl acetate (3×20 mL). The organic layer was separated and dried (Na₂SO₄), filtered, and evaporated in vacuo. Purification by column chromatography using DCM/MeOH gradient (20:1, 10:1) provided the title compound.

General Synthetic Procedure for Thioamide Bioisostere Derivatives. To a solution of (S)-2-phenyl-2-(4-thioxo-1,4-dihydro-5H-pyrazolo[3,4-d]pyrimidin-5-yl) acetic acid (14) (200, 0.7 mmol) in DMF (5 mL) were added (Benzotriazol-1-yloxy)tris(dimethylamino)phosphonium hexafluorophosphate (370 mg, 0.84 mmol), N,N-diisopropylethylamine (245 μL, 1.4 mmol) and the respective substituted amine (0.7 mmol). The reaction mixture was stirred at room temperature overnight. Water was added to the reaction mixture and was extracted with ethyl acetate (3×20 mL). The organic layer was separated and dried (Na₂SO₄), filtered, and evaporated in vacuo. Purification by column chromatography using DCM/MeOH gradient (20:1, 10:1) provided the title compound.

### Example 1: Synthesis of Compound 1

1-(azepan-1-yl)-2-phenyl-2-(4-thioxo-1,4-dihydro-5H-pyrazolo[3,4-d]pyrimidin-5-yl)ethan-1-one (1). According to the general procedure for thioamide bioisostere derivatives, the title compound was obtained. Rr: 0.3 (DCM: MeOH, 10:1). ¹H NMR (400 MHZ, CDCl₃): δ_{H} 11.31 (1H, br s), 8.36 (1H, s), 8.30 (1H, s), 8.05 (1H, s), 7.46-7.40 (5H, m), 3.86-3.81 (1H, m), 3.72-3.68 (1H, m), 3.54-3.48 (1H, m), 3.39-3.37 (1H, m), 2.04-1.95 (1H, m), 1.85-1.77 (1H, m), 1.73-1.58 (5H, m), 1.49-1.40 (1H, m). ¹³C NMR (100 MHZ, CDCl₃): δ_{C} 180.86, 166.72, 149.78, 146.37, 139.24, 134.18, 129.91, 129.13, 117.48, 65.54, 48.18, 46.65, 28.39, 27.62, 27.32, 26.51. HRMS-ESI (m/z): [M+H]⁺ calcd for C₁₉H₂₂N₅OS, 368.1540; found 368.1549.

### Example 2: Synthesis of Compound 13a

5-(2-(azepan-1-yl)-2-oxo-1-phenylethyl)-1,5-dihydro-4H-pyrazolo[3,4-d]pyrimidin-4-one (13a). According to the general procedure for amide bioisostere derivatives, the title compound was obtained. R_{f}: 0.3 (DCM/MeOH, 10:1). ¹H NMR (400 MHZ, CD₃OD): δ_{H} 8.20 (1H, br s), 7.71 (1H, s), 7.54-7.51 (3H, m), 7.46 (2H, t, J=6.0 Hz), 7.08 (1H, s), 3.86-3.83 (1H, m), 3.66-3.62 (m, 1H), 3.45-3.39 (1H, m), 3.37-3.30 (1H, m), 1.99-1.90 (1H, m), 1.84-1.74 (1H, m), 1.73-1.51 (5H, m), 1.49-1.41 (1H, m). ¹³C NMR (100 MHZ, CD₃OD): δ_{C} 167.94, 149.04, 133.10, 129.67, 129.63, 129.29, 104.36, 57.55, 46.51, 28.05, 26.93, 26.56, 25.99. HRMS-ESI (m/z): [M+H]⁺ calcd for C₁₉H₂₂N₅O₂, 352.1768; found, 352.1791. HPLC: retention time, 3.894 min; purity, 99%.

### Example 3: Synthesis of Compound 13b

5-(2-(4-hydroxypiperidin-1-yl)-2-oxo-1-phenylethyl)-1,5-dihydro-4H-pyrazolo[3,4-d]pyrimidin-4-one (13b). According to the general procedure for amide bioisostere derivatives, the title compound was obtained. R_{f}: 0.2 (DCM: MeOH, 10:1). ¹H NMR (400 MHZ, DMSO-D₆): δ_{H} 13.90 (1H, br s), 7.65-7.62 (1H, br d), 7.52-7.40 (5H, m), 7.10 (1H, s), 4.80-4.76 (1H, br d), 4.07-3.94 (1H, m), 3.67-3.55 (2H, m), 3.27-3.02 (2H, m), 1.73 (1H, br s), 1.57-1.20 (2H, m), 1.22-0.85 (1H, m). ¹³C NMR (100 MHZ, DMSO-D₆): δ_{C} 165.02, 165.90, 157.33, 149.20, 134.25, 134.18, 130.13, 129.83, 129.70, 104.66, 65.69, 65.52, 56.73, 49.08, 43.14, 40.58, 40.28, 39.33, 34.53, 34.21, 34.11. HRMS-ESI (m/z): [M+H]⁺ calcd for C₁₈H₂₀N₅O₃, 354.1561; found, 354.1562.

### Example 4: Synthesis of Compound 14b

1-(4-hydroxypiperidin-1-yl)-2-phenyl-2-(4-thioxo-1,4-dihydro-5H-pyrazolo[3,4-d]pyrimidin-5-yl)ethan-1-one (14b). According to the general procedure for thioamide bioisostere derivatives, the title compound was obtained. R_{f}: 0.2 (DCM: MeOH, 10:1). ¹H NMR (400 MHz, DMSO-d₆): δ_{H} 14.17 (1H, br s), 8.26 (2H, d, J=2.8 Hz), 7.90 (1H, d, J=12.4 Hz), 7.56-7.48 (3H, m), 7.41-7.36 (2H, m), 4.82-4.78 (1H, m), 4.05-3.99 (1H, m), 3.69-3.54 (2H, m), 3.32-3.03 (2H, m), 1.79-1.58 (2H, m), 1.51-0.95 (2H, m). ¹³C NMR (100 MHZ, DMSO-d₆): δ_{C} 164.88, 149.32, 138.88, 134.48, 134.40, 130.32, 130.09, 130.00, 129.54, 129.41, 117.02, 65.72, 65.43, 61.62, 65.43, 61.62, 61.59, 43.31, 43.02, 40.33, 34.77, 34.39, 34.27, 34.09. HRMS-ESI (m/z): [M+H]⁺ calcd for C₁₈H₂₀N₅O₂S, 370.1332; found, 370.1335.

### Example 5: Synthesis of Compound 13c

5-(2-(3-hydroxypiperidin-1-yl)-2-oxo-1-phenylethyl)-1,5-dihydro-4H-pyrazolo[3,4-d]pyrimidin-4-one (13c). According to the general procedure for amide bioisostere derivatives, the title compound was obtained. R_{f}: 0.2 (DCM: MeOH, 10:1). ¹H NMR (500 MHZ, CD₃OD): δ_{H} 8.21 (1H, s), 7.76-7.68 (1H, m), 7.55-7.41 (5H, m), 7.23-7.15 (1H, m), 4.38-3.95 (1H, m), 3.88-3.65 (1H, m), 3.64-3.35 (1H, m), 3.25-2.71 (2H, m), 1.95-1.61 (2H, m), 1.59-1.43 (2H, m). ¹³C NMR (125 MHZ, CD₃OD): δ_{C} 166.96, 166.81, 166.79, 158.21, 158.08, 153.89, 149.20, 149.09, 149.03, 133.72, 133.49, 133.30, 133.18, 132.91, 129.72, 129.70, 129.65, 129.58, 129.52, 129.48, 129.28, 129.17, 129.10, 104.39, 66.18, 65.42, 65.24, 65.22, 57.53, 57.38, 57.36, 57.19, 52.54, 51.91, 48.94, 45.86, 45.43, 42.77, 42.67, 32.80, 32.10, 31.99, 31.73, 22.90, 22.44, 22.11, 21.82. HRMS-ESI (m/z): [M+H]⁺ calcd for C₁₈H₂₀N₅O₃, 354.1561; found, 354.1566.

### Example 6: Synthesis of Compound 14c

1-(3-hydroxypiperidin-1-yl)-2-phenyl-2-(4-thioxo-1,4-dihydro-5H-pyrazolo[3,4-d]pyrimidin-5-yl)ethan-1-one (14c): According to the general procedure for thioamide bioisostere derivatives, the title compound was obtained. R_{f}: 0.2 (DCM: MeOH, 10:1). ¹H NMR (400 MHZ, CD₃OD): δ_{H} 8.37-8.34 (1H, m), 8.31 (1H, br s), 7.95-7.88 (1H, m), 7.53-7.48 (3H, m), 7.46-7.367 (2H, m), 4.41-4.23 (1H, m), 4.06-3.82 (1H, m), 3.77-3.59 (1H, m), 2.10-1.79 (1H, m), 1.71-1.66 (1H, m), 1.29-1.01 (1H, m). ¹³C NMR (100 MHZ, CD₃OD): δ_{C} 181.52, 166.19, 166.13, 165.93, 149.04, 148.93, 148.88, 147.21, 137.01, 133.90, 133.82, 133.58, 129.75, 129.67, 129.58, 129.53, 129.08, 129.03, 128.99, 128.87, 116.94, 66.33, 65.48, 65.33, 62.12, 62.02, 61.90, 61.76, 52.57, 51.93, 48.94, 48.85, 45.87, 45.41, 42.73, 42.46, 32.83, 32.09, 31.86, 23.02, 22.55, 22.41, 22.29. HRMS-ESI (m/z): [M+H]⁺ calcd for C₁₈H₂₀N₅O₂S, 370.1332; found, 370.1335.

### Example 7: Synthesis of Compound 13d

5-(2-(4-methoxypiperidin-1-yl)-2-oxo-1-phenylethyl)-1,5-dihydro-4H-pyrazolo[3,4-d]pyrimidin-4-one (13d): According to the general procedure for amide bioisostere derivatives, the title compound was obtained. R_{f}: 0.2 (DCM: MeOH, 10:1). ¹H NMR (400 MHZ, CDCl₃): δ_{H} 12.05 (1H, br s), 8.18 (1H, s), 7.91 (1H, s), 7.51-7.44 (m, 3H), 7.51-7.44 (3H, m), 7.41-7.39 (3H, m), 7.20 (1H, s), 3.82-3.56 (6H, m), 3.42-3.33 (2H, m). ¹³C NMR (100 MHZ, CDCl₃): δ_{C} 165.88, 157.53, 157.38, 152.82, 149.85, 135.92, 133.88, 133.53, 129.86, 129.84, 129.69, 129.66, 129.06, 129.03, 104.65, 74.73, 74.42, 56.28, 56.07, 55.76, 55.66, 42.77, 42.64, 39.76, 39.56, 30.41, 30.31, 30.02, 29.90. HRMS-ESI (m/z): [M+H]⁺ calcd for C₁₉H₂₂N₅O₃, 368.1717; found, 368.1745.

### Example 8: Synthesis of Compound 14d

1-(4-methoxypiperidin-1-yl)-2-phenyl-2-(4-thioxo-1,4-dihydro-5H-pyrazolo[3,4-d]pyrimidin-5-yl)ethan-1-one (14d): According to the general procedure for thioamide bioisostere derivatives, the title compound was obtained. R_{f}: 0.2 (DCM: MeOH, 10:1). ¹H NMR (400 MHZ, MeOD) δ_{H} 1.23-1.33 (1H, m, CH), 1.35-1.44 (1H, m, CH), 1.50-1.86 (2H, m, CH), 3.06-3.16 (1H, m, CH), 3.25 (3H, s, CH₃), 3.27-3.64 (3H, m, CH), 3.72-3.87 (1H, m, CH), 7.12-7.18 (1H, m, CH), 7.26-7.33 (2H, m, ArCH), 7.35-7.45 (3H, m, ArCH), 7.78 (1H, m, ArCH), 8.07-8.13 (1H, m, ArCH). ¹³C NMR (100 MHZ, MeOD) δ_{C} 29.92, 30.40, 30.51, 39.58, 39.77, 55.76, 55.87, 56.20, 129.04, 129.98, 136.31, 150.17, 157.37, 165.81. ESI-HRMS (m/z): [M+H]⁺ calcd for C₁₉H₂₁N₅O₂S, 384.1465; found, 384.1479.

### Example 9: Synthesis of Compound 13e

5-(2-morpholino-2-oxo-1-phenylethyl)-1,5-dihydro-4H-pyrazolo[3,4-d]pyrimidin-4-one (13e): According to the general procedure for amide bioisostere derivatives, the title compound was obtained. R_{f}: 0.2 (DCM: MeOH, 10:1). ¹H NMR (400 MHZ, CDCl₃): δ_{H} 11.10 (1H, br s), 8.18 (1H, s), 7.91 (1H, s), 7.51-7.44 (3H, m), 7.41-7.39 (2H, m), 7.20 (1H, s), 3.82-3.56 (6H, m), 3.42-3.33 (2H, m).¹³C NMR (100 MHZ, CDCl₃): δ_{C} 166.35, 157.44, 152.79, 149.72, 135.96, 133.30, 129.99, 129.89, 129.07, 104.65, 66.63, 66.16, 56.14, 46.13, 42.94. HRMS-ESI (m/z): [M+H]⁺ calcd for C₁₇H₁₈N₅O₃, 340.1404; found, 340.1424.

### Example 10: Synthesis of Compound 14e

1-morpholino-2-phenyl-2-(4-thioxo-1,4-dihydro-5H-pyrazolo[3,4-d]pyrimidin-5-yl)ethan-1-one (14e): According to the general procedure for thioamide bioisostere derivatives, the title compound was obtained. R_{f}: 0.2 (DCM: MeOH, 10:1). ¹H NMR (500 MHZ, CD₃OD): δ_{H} 8.34 (2H, s), 8.02 (1H, s), 7.48-7.46 (3H, m), 7.40-7.38 (2H, m), 6.01 (1H, br s), 3.83-3.65 (6H, m), 3.51-3.47 (1H, m), 3.39-3.34 (1H, m). 3C NMR (125 MHZ, CD₃OD): δ_{C} 180.98, 165.62, 149.67, 139.04, 133.38, 130.13, 130.03, 129.05, 66.75, 66.39, 61.19, 46.18, 43.02. HRMS-ESI (m/z): [M+H]⁺ calcd for C₁₇H₁₈N₅O₂S, 356.1176; found, 356.1179.

### Example 11: Synthesis of Compound 13f

5-(2-(3-hydroxypyrrolidin-1-yl)-2-oxo-1-phenylethyl)-1,5-dihydro-4H-pyrazolo[3,4-d]pyrimidin-4-one (13f): According to the general procedure for amide bioisostere derivatives, the title compound was obtained. R_{f}: 0.2 (DCM: MeOH, 10:1). ¹H NMR (400 MHZ, DMSO-D₆): δ_{H} 13.90 (1H, br s), 8.17 (1H, br s), 7.50-7.39 (5H, m), 6.94-6.82 (1H, m), 5.12-4.93 (1H, m), 5.12-4.93 (1H, m), 5.12-4.93 (1H, m), 4.33-4.09 (1H, m), 3.76-3.43 (2H, m), 3.19-2.93 (2H, m), 2.0-1.76 (2H, m). ¹³C NMR (100 MHZ, DMSO-D₆): δ_{C} 166.16, 156.91, 152.76, 149.32, 136.09, 134.15, 130.14, 130.09, 129.74, 129.70, 129.58, 129.49, 129.42, 104.44, 69.91, 69.80, 68.11, 68.05, 57.48, 57.22, 55.34, 55.07, 54.84, 54.70, 54.55, 49.07, 44.85, 44.68, 44.61, 44.47, 34.27, 34.23, 32.56, 32.56. HRMS-ESI (m/z): [M+H]⁺ calcd for C₁₇H₁₈N₅O₃, 340.1404; found, 340.1407.

### Example 12: Synthesis of Compound 13g

5-(2-(3-hydroxyazetidin-1-yl)-2-oxo-1-phenylethyl)-1,5-dihydro-4H-pyrazolo[3,4-d]pyrimidin-4-one (13g): According to the general procedure for amide bioisostere derivatives, the title compound was obtained. R_{f}: 0.2 (DCM: MeOH, 10:1). ¹H NMR (400 MHZ, CD₃OD): δ_{H} 8.20 (1H, br s), 7.81 (1H, d, J=5.2 Hz), 7.50-7.44 (5H, m), 6.73 (1H, s), 4.65-4.56 (2H, m), 4.56 (1H, br d), 4.37-4.35 (1H, m), 4.25-4.04 (1H, m), 3.94-3.87 (1H, m), 3.62-3.33 (1H, m). ¹³C NMR (100 MHZ, CD₃OD): δ_{C} 168.01, 167.84, 158.06, 148.59, 135.61, 132.61, 129.64, 129.58, 129.55, 129.09, 128.99, 104.42, 60.66, 60.47, 60.07, 59.88, 57.93, 56.18. HRMS-ESI (m/z): [M+H]⁺ calcd for C₁₆H₁₆N₅O₃, 326.1248; found, 326.1248.

### Example 13: Synthesis of Compound 14g

1-(3-hydroxyazetidin-1-yl)-2-phenyl-2-(4-thioxo-1,4-dihydro-5H-pyrazolo[3,4-d]pyrimidin-5-yl)ethan-1-one (14g). According to the general procedure for thioamide bioisostere derivatives, the title compound was obtained. R_{f}: 0.2 (DCM: MeOH, 10:1). ¹H NMR (500 MHZ, CD₃OD): δ_{H} 8.31 (1H, br d), 7.99 (1H, br d), 7.85 (1H, s), 7.52-7.41 (5H, m), 4.77-4.65 (1H, m), 4.60-4.33 (2H, m), 4.26-3.94 (1H, m), 3.92-3.67 (1H, m). ³C NMR (125 MHZ, CD₃OD): δ_{C} 181.41, 167.32, 167.08, 148.28, 147.18, 137.04, 133.33, 133.29, 129.67, 129.66, 129.56, 128.80, 128.76, 117.02, 60.70, 60.68, 60.63, 60.39, 60.16, 59.99, 59.76, 57.95, 57.90. HRMS-ESI (m/z): [M+H]⁺ calcd for C₁₆H₁₆N₅O₂S, 342.1019; found 342.1018.

### Example 14: Synthesis of Compound 13h

5-(2-Oxo-1-phenyl-2-thiomorpholinoethyl)-1,5-dihydro-4H-pyrazolo[3,4-d]pyrimidin-4-one (13h). According to the general procedure for amide bioisostere derivatives, the title compound was obtained. R_{f}: 0.2 (DCM/MeOH, 10:1). ¹H NMR (400 MHZ, CDCl₃): 0H 8.45 (1H, br s), 8.21 (1H, s), 7.94 (1H, s), 7.49 (3H, m), 7.41 (2H, m), 7.17 (1H, s), 4.11-4.06 (1H, m), 3.96-3.90 (1H, m), 3.85-3.80 (1H, m), 3.69-3.64 (1H, m), 2.76-261 (3H, m), 2.31-2.27 (1H, m). ¹³C NMR (100 MHZ, CDCl₃): δ_{C} 166.17, 157.22, 150.04, 135.97, 133.02, 130.12, 130.09, 129.24, 56.44, 48.67, 45.57, 27.38, 27.34. HRMS-ESI (m/z): [M+H]⁺ calcd for C₁₇H₁₈N₅O₂S, 356.1176; found, 356.1201.

### Example 15: Synthesis of Compound 14h

1-((3R,4S)-3,4-dihydroxypiperidin-1-yl)-2-phenyl-2-(4-thioxo-1,4-dihydro-5H-pyrazolo[3,4-d]pyrimidin-5-yl)ethan-1-one (14h). According to the general procedure for thioamide bioisostere derivatives, the title compound was obtained. R_{f}: 0.2 (DCM/MeOH, 10:1). ¹H NMR (400 MHZ, MeOD): 0H 8.42-8.31 (2H, m), 7.96-7.86 (1H, m), 7.51-7.38 (5H, m), 4.14-3.66 (3H, m), 3.63-3.25 (3H, m), 1.89-1.30 (2H, m). ¹³C NMR (100 MHZ, MeOD): δ_{C} 181.78, 166.36, 166.17, 149.04, 148.89, 143.17, 133.92, 133.82, 133.61, 129.77, 129.75, 129.68, 129.64, 129.52, 129.05, 128.95, 128.90, 128.86, 116.94, 116.91, 68.26, 68.18, 67.80, 67.76, 67.60, 67.52, 67.31, 67.17, 62.15, 61.95, 61.77, 61.65, 53.42, 46.78, 44.94, 43.98, 42.00, 41.10, 37.77, 37.47, 30.05, 29.60, 29.28, 29.19. ESI-HRMS (m/z): [M+H]⁺ calcd for C₁₈H₂₀N₅O₃S, 386.1281; found, 386.1294.

### Example 16: Synthesis of Compound 13i

5-(1-(4-chlorophenyl)-2-(3-hydroxypiperidin-1-yl)-2-oxoethyl)-1,5-dihydro-4H-pyrazolo[3,4-d]pyrimidin-4-one (13i). According to the general procedure for amide bioisostere derivatives, the title compound was obtained. ¹H NMR (400 MHZ, MeOD): δ_{H} 1.55 (2H, m, CH₂), 1.90 (2H, m, CH₂), 2.98 (1H, m, CH), 3.27 (1H, m, CH), 3.64 (1H, m, CH), 3.84 (1H, m, CH), 4.25 (1H, m, CH), 7.18 (1H, s, CH), 7.45 (2H, d, ArCH₂), 7.53 (2H, d, ArCH₂), 7.692 (1H, s, CH), 7.77 (1H, s, CH), 8.21 (1H, s, NH). ¹³C NMR (100 MHz; MeOD): δ_{C} 21.0, 30.9, 43.7, 46.4, 49.5, 55.6, 65.2, 130.5, 132.1, 136.0, 149.5, 157.3, 166.5. ESI-HRMS (m/z): [M−H]⁻ calcd for C₁₈H₁₈ClN₅O₃, 388.1183; found, 388.1176.

### Example 17: Synthesis of Compound 13j

5-(1-(4-fluorophenyl)-2-(3-hydroxypiperidin-1-yl)-2-oxoethyl)-1,5-dihydro-4H-pyrazolo[3,4-d]pyrimidin-4-one (13j). According to the general procedure for amide bioisostere derivatives, the title compound was obtained. ¹H NMR (400 MHZ, MeOD): δ_{H} 1.55 (2H, m, CH₂), 1.89 (2H, m, CH₂), 2.98 (1H, m, CH), 3.27 (1H, m, CH), 3.66 (1H, m, CH), 3.85 (1H, m, CH), 4.27 (1H, m, CH), 7.18 (1H, s, CH), 7.29 (2H, t, ArCH₂), 7.50 (2H, t, ArCH₂), 7.70 (1H, s, CH), 7.76 (1H, s, CH), 8.21 (1H, s, NH). ¹³C NMR (100 MHz; MeOD): δ_{C} 22.5, 29.2, 32.0, 42.7, 45.4, 48.9, 52.4, 56.5, 65.4, 66.0, 116.4, 131.6, 166.7. ESI-HRMS (m/z): [M−H]⁻ calcd for C₁₈H₁₈FN₅O₃, 372.1475; found, 372.1472.

### Example 18: Synthesis of Compound 13k

5-(1-(4-chlorophenyl)-2-(4-hydroxypiperidin-1-yl)-2-oxoethyl)-1,5-dihydro-4H-pyrazolo[3,4-d]pyrimidin-4-one (13k). According to the general procedure for amide bioisostere derivatives, the title compound was obtained. ¹H NMR (400 MHZ, MeOD): δ_{H} 1.50 (2H, m, CH₂), 1.89 (2H, m, CH₂), 3.16 (1H, m, CH), 3.30 (1H, m, CH), 3.69 (1H, m, CH), 3.85 (1H, m, CH), 4.14 (1H, m, CH), 7.16 (1H, s, CH), 7.44 (2H, t, ArCH₂), 7.55 (2H, t, ArCH₂), 7.73 (1H, s, CH), 7.73 (1H, s, CH), 8.15 (1H, s, NH). ¹³C NMR (100 MHz; MeOD): δ_{C} 29.3, 33.3, 33.6, 42.8, 56.5, 65.8, 129.8, 130.8, 131.7, 135.6, 166.2, 209.0. ESI-HRMS (m/z): [M−H]⁻ calcd for C₁₈H₁₈ClN₅O₃, 388.1177; found, 388.1176.

### Example 19: Synthesis of Compound 13l

5-(1-(4-fluorophenyl)-2-(4-hydroxypiperidin-1-yl)-2-oxoethyl)-1,5-dihydro-4H-pyrazolo[3,4-d]pyrimidin-4-one (13l). According to the general procedure for amide bioisostere derivatives, the title compound was obtained. ¹H NMR (400 MHZ, MeOD): δ_{H} 1.48 (2H, m, CH₂), 1.88 (2H, m, CH₂), 3.15 (1H, m, CH), 3.31 (1H, m, CH), 3.69 (1H, m, CH), 3.86 (1H, m, CH), 4.14 (1H, m, CH), 7.14 (1H, s, CH), 7.29 (2H, t, ArCH₂), 7.50 (2H, t, ArCH₂), 7.70 (1H, s, CH), 7.73 (1H, s, CH), 8.14 (1H, s, NH). ¹³C NMR (100 MHz; MeOD): δ_{C} 29.3, 33.1, 40.0, 42.9, 56.7, 65.9, 116.7, 131.6, 149.0, 162.1, 164.6, 166.3, 209.3. ESI-HRMS (m/z): [M−H]⁻ calcd for C₁₈H₁₈FN₅O₃, 372.1467; found, 372.1472.

### Biological Assays

### General Methods

Cell Culture. The cell line SH-SY5Y was cultured in DMEM/F12 media supplemented with 10% fetal bovine serum, 1% penicillin-streptomycin and maintained as monolayer cultures in a humidified atmosphere containing 5% CO₂ at 37° C.⁴² All cell lines were authenticated via short tandem repeat analysis and tested for mycoplasma using the a commercial mycoplasma detection kit as per the manufacturer's instructions, showing no contamination. Where indicated, cells were also cultured in DMEM/low glucose media. All compounds were diluted to 20 mM solution in DMSO and were serially diluted in cell culture media for cell treatments to a final concentration range of 0.01 to 100 μM, maintaining the final DMSO concentration at less than 1%. Positive control compound HupA was serially diluted in a similar fashion as the synthesized compounds.

Amyloid Preparation. The peptide Aβ₁₋₄₂, referred to as “Aβ”, was obtained from a commercial source. The oligomers of Aβ were prepared using an established method,⁴³ Aβ peptide was resuspended in 0.5 mM NaOH at a concentration of 350 mM and stored at −80° C. For use in cell cultures, the stock solution was incubated at 37° C. for 5-7 days. Before use, the peptide was diluted to 25 μM in DMEM/low glucose media.

Estradiol ELISA assay. SH-SY5Y cells were treated with compounds (24 hours), followed by Aβ (72 hours) in a similar fashion to that employed in the cell viability assays, the cell culture medium was collected, and cells were lysed using RIPA buffer and protease inhibitor cocktail, then proteins were quantified via BCA assay. An estradiol assay was performed. In brief, the plate was loaded with samples, along with the estradiol tracer and the specific antiserum to estradiol and incubated for one hour at room temperature. After five washing steps, Ellman's Reagent was added, and the plate developed for 60 minutes with gentle shaking at room temperature. The calculated estradiol concentration was normalized to the total protein content of the samples.

Cell Viability Assays. The SH-SY5Y cells were plated at a density of 50,000 cells/well in 96-well plates and allowed to adapt overnight in a humidified atmosphere containing 5% CO₂ at 37° C. The cells were pretreated (24 hours) with the test compound and exchanged to a serum-free DMEM/low glucose media, prior to incubation with 25 μM of Aβ. Cell viability was measured after 48 hours using commercial MTS assay or lactate dehydrogenase (LDH) cytotoxicity assay.

Mitochondrial Stress Test. Cultured SH-SY5Y cells were plated at a density of 50,000 cells/well in 24-well assay plates and allowed to adapt for 24 hours days prior to pretreatment (24 hours) with the test compound followed by incubation with 25 μM of Aβ. After 48 hours, media was replaced with assay medium consisting of XF Base Medium supplemented with 10 mM glucose, 10 mM pyruvic acid, and 1 mM L-glutamine. Subsequently, the analysis of mitochondrial oxygen consumption rate (OCR) was performed in a flux analyzer. The OCR values were obtained both during baseline (prior to addition of any Mito Stress Test substances), and after the addition of 1.5 M oligomycin, 2 μM FCCP and 0.5 μM rotenone+0.5 μM antimycin A, respectively. Prior to analysis, data were corrected by withdrawing non-mitochondrial respiration (measured after the injection of rotenone and antimycin A) from all measured OCR values. After the experiment, cells were lysed using RIPA buffer and protease inhibitor cocktail, then proteins were quantified via BCA assay. Results were normalized to protein concentration of each well to its OCR value.

Respiration Measurements (5XFAD cortical neurons). Primary cortical neurons were plated on poly-D-lysine-coated Seahorse XF24 cell culture microplates at a density of 3×10⁴ cells/well and cultured for 14 days. At DIV (day in vitro) 12, neurons were treated with indicated compound for 48 hours. On day of the experiment, cells were washed three times and pre-incubated for 1 h in Assay Media with 10 mM Glucose, 1 mM Pyruvate, and 2 mM L-Glutamine. Measurement of intact cellular respiration was performed using an analyzer and a commercial Mito Stress Test Kit. Respiration was measured under basal conditions, and in response to 1.5 μM oligomycin followed by the addition of the electron transport chain accelerator ionophore 4-(trifluoromethoxy)phenylhydrazone (FCCP, 1 μM) which induces maximal OCR (Oxygen Consumption Rate). Finally, respiration was stopped by adding the electron transport chain inhibitors rotenone and antimycin A (0.5 μM). Values were normalized to cellular protein levels.

Immunofluorescent Microscopy and Image Analysis. Isolated primary mouse cortical neurons from the 5XFAD Alzheimer's Disease mouse model were cultured for 10 days and then treated with 14b at 10 or 50 μM for 48 hours. Primary neurons growing on the coverslips were washed three times with PBS and fixed with 4% PFA. After permeabilization with 0.5% Triton™ X-100 for 15 minutes, coverslips were blocked with 10% NGS for 30 min. Coverslips were rinsed with 1% NGS in PBS and incubated with anti-TOM20 antibody at 4° C. overnight. Coverslips were then rinsed with 1% NGS, blocked in 10% NGS for 10 min, and rinsed with 1% NGS. Coverslips were incubated with species-specific AlexaFluor® 488-conjugated Abs diluted in 1% NGS for 2 h at room temperature in the dark. Coverslips were rinsed 3× with PBS. Excess liquid was removed, and slides were coverslipped using Fluoromount-G™ mounting medium. Slides were imaged using a confocal microscope with 63x/1.4NA Oil objective. The acquired images were processed for background subtraction in Zen software. Mitochondrial length was calculated from single plane images. Mitochondrial length was defined as the Feret maximum.

Statistical Analyses. The IC₅₀ values of each compound were acquired from a single experiment with each inhibitor concentration run in triplicate and the statistical significance was calculated using the Student t test, or a one-way or two-way ANOVA. A P value of <0.05 was considered statistically significant. IC₅₀ values were calculated.

### Example 20: Quantification of Estradiol as Measurement for ABAD Activity

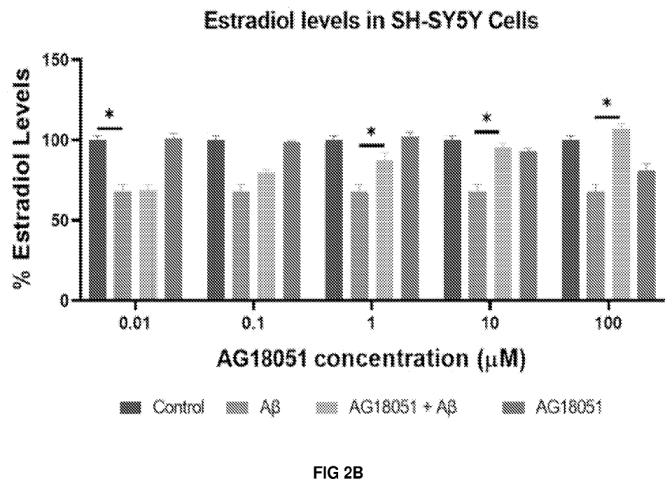

To determine the effect of Aβ on estradiol production, and ABAD's role in this process, wildtype human SH-SY5Y cell lines, which are known to express ABAD, were incubated for 72 hours with Aβ.²⁰ Estradiol levels in the cell lysate were measured with a commercial ELISA-based estradiol assay. In accordance with published literature,²⁰ estradiol levels were significantly decreased after Aβ exposure (FIG. 2A). To determine if AG18051 treatment affects Aβ-induced reduction of estradiol levels, 0.1 or 1 μM AG18051 was preincubated in SH-SY5Y cells for 24 hours prior to Aβ exposure. The Aβ-ABAD PPI inhibitor significantly maintained estradiol levels in the cells when compared to Aβ-only control in a dose-dependent manner (FIG. 2A). The effect of HupA, a clinically approved AD treatment in China,³² which has recently been shown to possess ABAD inhibition activity,³³ at 10 AM concentration was determined (FIG. 2A). AG18051 at 1 μM protected SH-SY5Y cells from Aβ-induced reduction of estradiol production to a greater significance than HupA. This confirms literature reports that AG18051 is neuroprotective and that it exerts this effect by ameliorating Aβ-induced reduction of estradiol production by ABAD.^{20, 23}

This assay methodology allows direct screening of compound activity in the presence of Aβ and provides direct evidence of the ability of compounds to rescue Aβ-induced reduction of estradiol production by ABAD. To measure the effect of AG18051 alone in SH-SY5Y cell lines, a 0.01-100 μM dose-response range was tested, and their effect on estradiol levels (FIG. 2B). Contrary to a previous report that demonstrated reduction in estradiol levels and toxicity with concentrations higher than 0.1 μM,²⁰ no significant reduction in estradiol levels was observed. However, at 100 μM, found AG18051 was found to induce a 20% decrease in estradiol levels when compared to control. To evaluate the effect of the increase of AG18051 dose in maintaining estradiol levels reduced by Aβ, AG18051 was tested in increasing concentrations in the presence of Aβ (FIG. 2B). AG18051 was found to display a dose-dependent increase in estradiol levels. Without wishing to be bound by theory, this suggests that the Aβ-ABAD PPI may be inhibited by AG18051, as no significant change in estradiol levels was seen with AG18051 alone; alternatively, this may also suggest that the Aβ-ABAD PPI leads to a feedback mechanism due to the enzyme's reductive activity and ABAD expression is increased leading overall decreased estradiol levels.

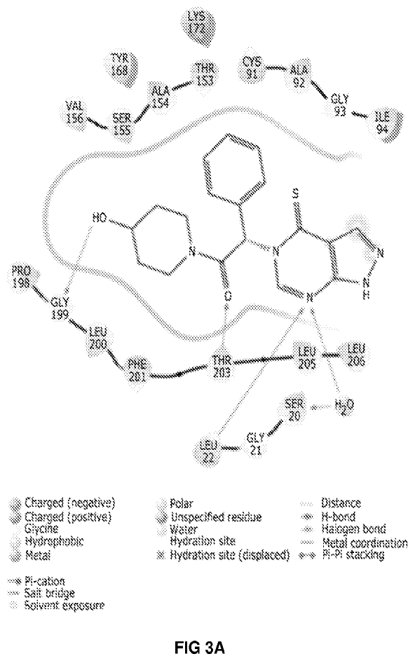

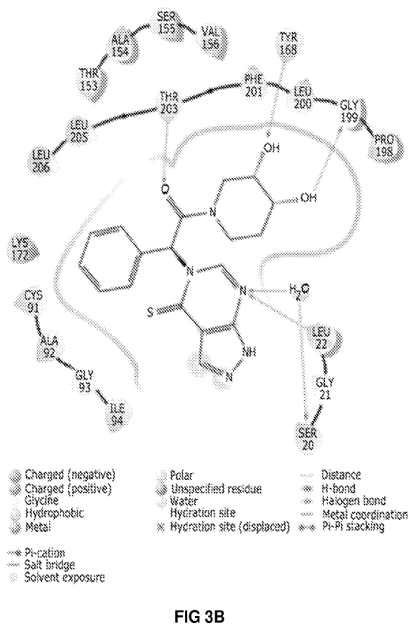

Synthesized ABAD derivatives were screened in the estradiol assay and the parent compound AG18051 was used as positive control (Table 2, below). A comparison of activity between thioamide derivatives of the parent compound and amide bioisosteres was of particular interest based on molecular modeling data that did not predict a crucial role for the thioamide thione (FIGS. 3A and 3B). The amide bioisostere of AG18051 (13a) proved to have similar inhibition activity to the parent compound 1 with IC₅₀s of 1.093 and 1.224 μM, respectively. Comparison between the amide and thioamide bioisostere in compounds possessing a wide variety of substitutions around the heterocylic ring revealed equipotent attenuation of Aβ-induced reduction of estradiol (compare 13b-13e, 14b-14e).

Table 2 below shows the results of the estradiol assay.

TABLE 2

| Compound No. | IC₅₀ (μM) |
|---|---|
| 1 | 1.224 ± 0.050 |
| 13a | 1.093 ± 0.064 |
| 13b | 0.591 ± 0.092 |
| 14b | 0.738 ± 0.105 |
| 13c | 0.707 ± 0.055 |
| 14c | 0.732 ± 0.089 |
| 13d | 0.735 ± 0.068 |
| 14d | 0.653 ± 0.061 |
| 13e | 2.484 ± 0.096 |
| 14e | 3.574 ± 0.102 |
| 13f | 1.158 ± 0.904 |
| 13g | 1.442 ± 0.092 |
| 14g | 2.631 ± 0.042 |
| 13h | 5.395 ± 0.111 |
| 14h | 0.898 ± 0.099 |
| 13i | 3.159 ± 0.249 |
| 13j | 2.270 ± 0.025 |
| 13k | 5.018 ± 0.292 |
| 131 | 2.293 ± 0.221 |

### Example 21: Structure-Activity Relationship Study

Having established no clear preference for the thioamide or amide, structural features were explored, including substituents around, and contraction of, the azepane ring. Contraction of the seven-membered azepane ring to a six-membered piperidine ring bearing a 3-(13c, 14c) or 4-substituted hydroxy group (13b, 14b) resulted in compounds with greater activity (IC₅₀=0.71, 0.73, 0.59 and 0.74 μM respectively). Introduction of a methoxy at the 4-position of the piperidine ring (13d, 14d), provided similarly equipotent compounds (IC₅₀'s=0.74 and 0.65 μM respectively), suggesting the importance of a hydrogen bond accepting group in this area of the compound.

When the piperidine ring is replaced with a morpholine or thiomorpholine (13e, 14e) to bring the hydrogen bond acceptor moiety into the ring, activity is reduced by 3-5-fold (IC₅₀=2.5, 3.6 μM respectively), suggesting an extracyclic electronegative moiety is favorable. Truncation in ring size from piperidine (n=6) to pyrrolidine (n=5) to azetidine (n=4) (13f, 13g and 14g respectively), results in increasingly reduced potency, potentially indicating the formation of a weaker hydrogen bond by increasing the distance between donor and acceptor.

In silico modelling was employed to understand predicted interactions between the synthesized compounds and ABAD (FIGS. 3A and 3B). These studies predict key hydrogen bonding interactions between the terminal alcohol of 14b and Glyl99, between the amide carbonyl and Thr203 and between the unsubstituted nitrogen in the pyrimidine ring and Leu22 via a water bridge (FIG. 3A). These interactions are conserved in all modeled compounds. Compound 14b was predicted to possess an affinity of −5.82 kcal/mol which corresponds well to its experimentally calculated IC₅₀ of 0.74 μM.

The addition of a second alcohol group to the piperidine ring moiety (3,4-dihydroxypiperidine) was predicted to engage in hydrogen bonding with the Tyr168 residue, while retaining the 4-hydroxy interaction with Glyl99, accounting for a higher predicted affinity of −6.06 kcal/mol (FIG. 3B). However, when this derivative (14h) was synthesized, a slightly attenuated IC₅₀ of 0.90 μM was observed. Gratifyingly, the in silico data supported the obtained screening data with no predicted interactions between either the ring carbonyl or ring thione and any amino acid residue. Thus, compounds 13b (carbonyl) IC₅₀=0.591±0.092 and compound 14b (thione) IC₅₀=0.738±0.105 are equipotent within standard measurement of error.

Next, modifications were explored on the phenyl ring of the parent scaffold. Halogenation at the 4-position with chlorine (13i and 13k) or fluorine (13j and 13l) significantly attenuated activity with obtained IC₅₀ values of 3.16, 5.0, 2.27 and 2.3 μM respectively, albeit with fluorine affording slightly more active compounds. However, the unsubstituted phenyl ring provides far greater activity.

### Example 22: Inhibitors of the Aβ-ABAD PPI Ameliorate Aβ-Induced Toxicity

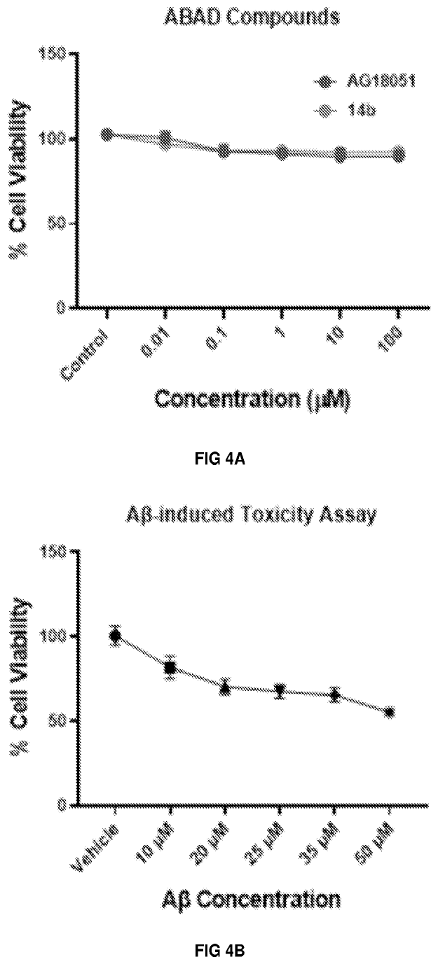

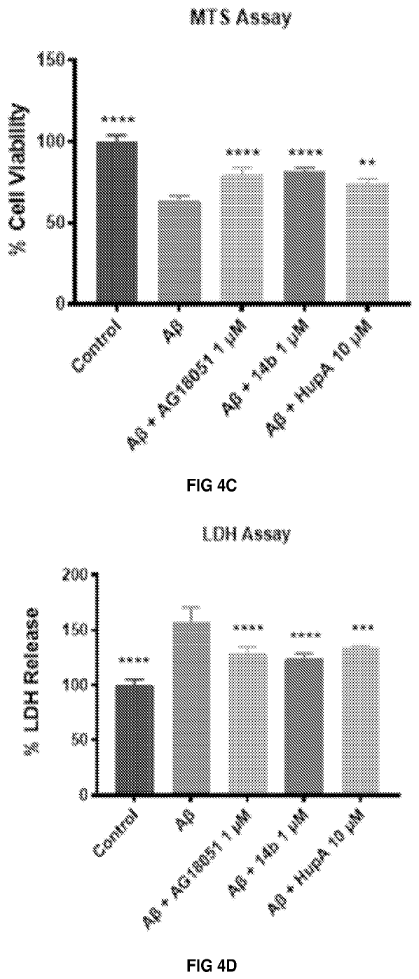

Employing human neuroblastoma SH-SY5Y ‘neuron-like’ cells, known to express ABAD,²⁰ experimental conditions are described herein that result in reproducible loss of 65% cell viability upon treatment with 25 μM Aβ after 48-hour incubation (FIG. 4A). Compound 1 (AG18051) and its analog 14b were screened for toxic effects in the SH-SY5Y cell lines. Both compounds displayed no toxicity to reduce cell viability up to concentrations of 100 μM (FIG. 4B).

Compounds 1 and 14b significantly ameliorate Aβ-induced toxicity in SH-SY5Y cells, affording 79% and 82% protection of cell viability at 1 μM concentration respectively, when measured by MTS assay (FIG. 4C). HupA at 10 μM concentration provided 74% protection from Aβ-induced toxicity. Employing a lactate dehydrogenase (LDH) release assay as a secondary measurement of cell viability, protective effects were confirmed with both 1 and 14b significantly reducing LDH release compared with Aβ treatment alone (FIG. 4D). Again, synthesized compound 14b at 1 μM, afforded greater protection than HupA at 10 μM concentration.

This data represents critical findings for continued design of inhibitors; azepane ring derivatives are not readily available and involve significant synthetic steps to generate. Whereas piperidine ring derivatives are much easier to synthesize and therefore allow for greater exploration of chemical space around this compound fragment. Both compounds 14b and 14c share a similar positioning of the hydroxyl group, being two carbons distant from the nitrogen within the ring system. This would place the alcohol moiety in a similar spacial arrangement in both compounds, potentially engendering greater binding. Collectively, this data shows that a large degree of lipophilicity at this fragment of the parent compound is not required for ABAD inhibition activity to the degree that was first reported for AG18051.³⁴

### Example 23: Neuroprotective Aβ-ABAD PPI Inhibitors Rescue Aβ-Induced Mitochondrial Dysfunction in SH-SY5Y Cells

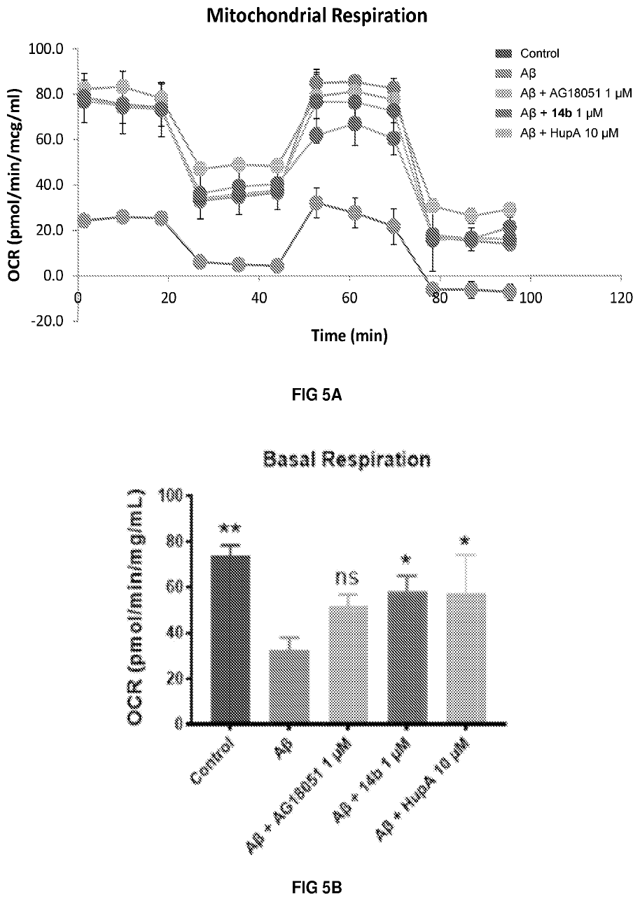

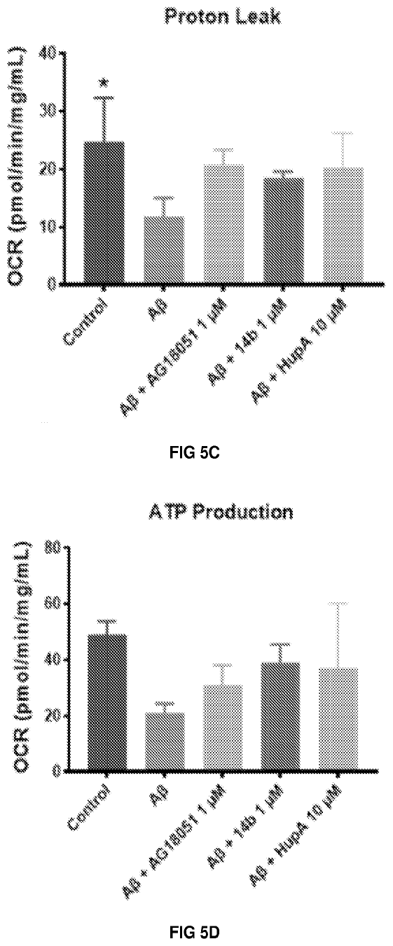

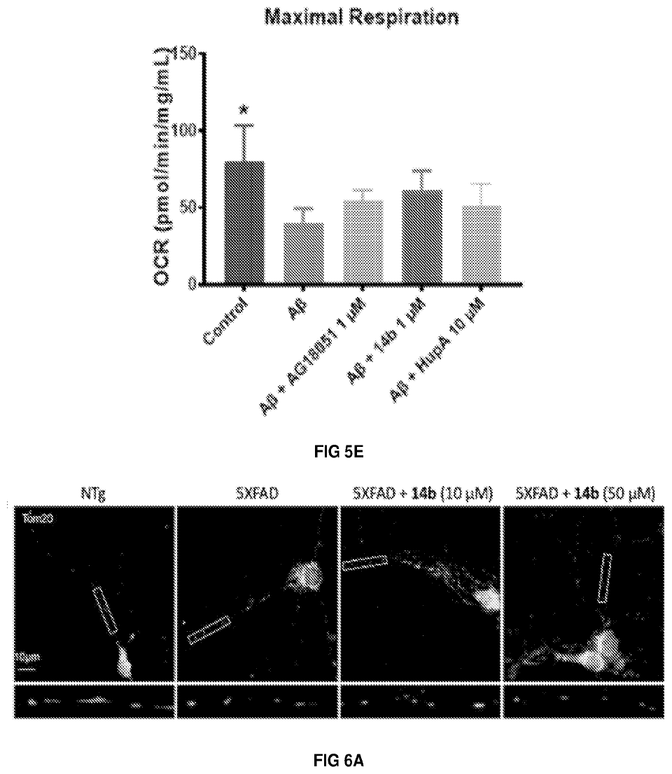

Given the reported mitochondrial dysfunction in AD,³⁵⁻³⁷ the effect of compound 14b to rescue the depleted mitochondrial respiration of Aβ-treated SH-SY5Y cells was determined using an extracellular flux analyzer (FIG. 5A). In agreement with reported literature showing dysfunction in mitochondrial function,^{38, 39} basal respiration in Aβ-treated cells was significantly lower than control cells (67%; FIG. 5B). This level was increased, but not significantly, by the pretreatment of SH-SY5Y cells with 1 (1 μM) restoring 37% of the cell respiration reduced by AB, while the respiration level was significantly increased by 14b (at 1 μM), restoring 46%. The positive control HupA (10 μM) was also able to restore respiration levels by 46%. Similar effects were observed in proton leak (FIG. 5C), ATP production (FIG. 5D) and maximal respiration measurements (FIG. 5E). The observed mitochondrial dysfunction is consistent with other reports of SH-SY5Y treated with Aβ or overexpressing APP.^{40, 41} Cells treated with Aβ showed a significant reduction in proton leak, ATP production and maximal respiration, 55%, 60% and 50%, respectively, when compared to control. Treatments with Compound 1 (1 μM), 14b (1 M) or HupA (10 μM) showed a trend to restore this lost mitochondrial function induced by AB.

This data shows that Compound 1 but with greater efficiency, 14b, can successfully rescue Aβ-induced mitochondrial dysfunction. Basal respiration, proton leak, ATP production and maximal respiration measurements were reduced with Aβ treatment. These functions were restored with 1 μM pretreatment of 14b. A similar effect was observed with HupA, but at 10 μM concnetration, demonstrating that our synthesized compound provides greater effect than the clinically approved AD treatment in China, HupA.³²

### Example 24: Neuroprotective Aβ-ABAD PPI Inhibitor 14b Rescues Defective Mitochondrial Morphology in Isolated 5XFAD Mouse Model Cortical Neurons

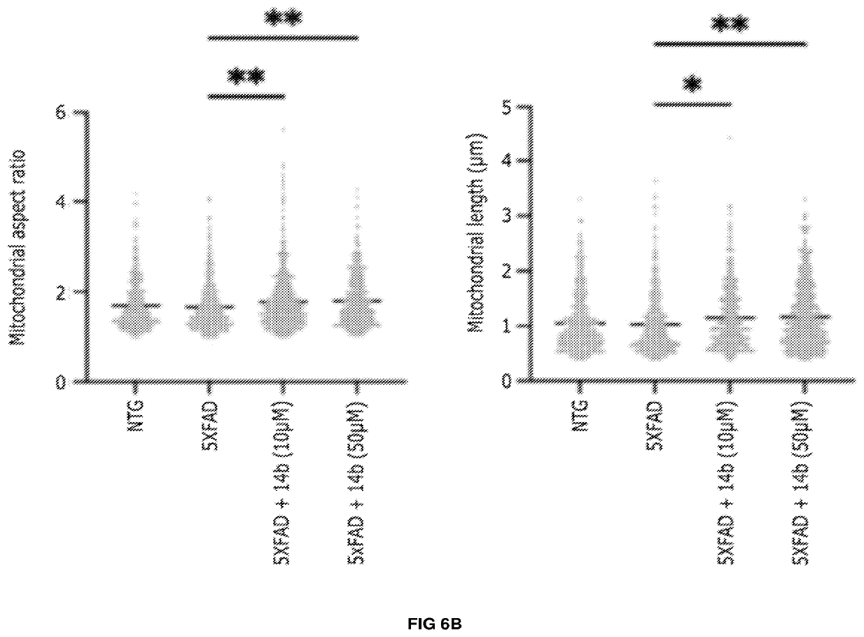

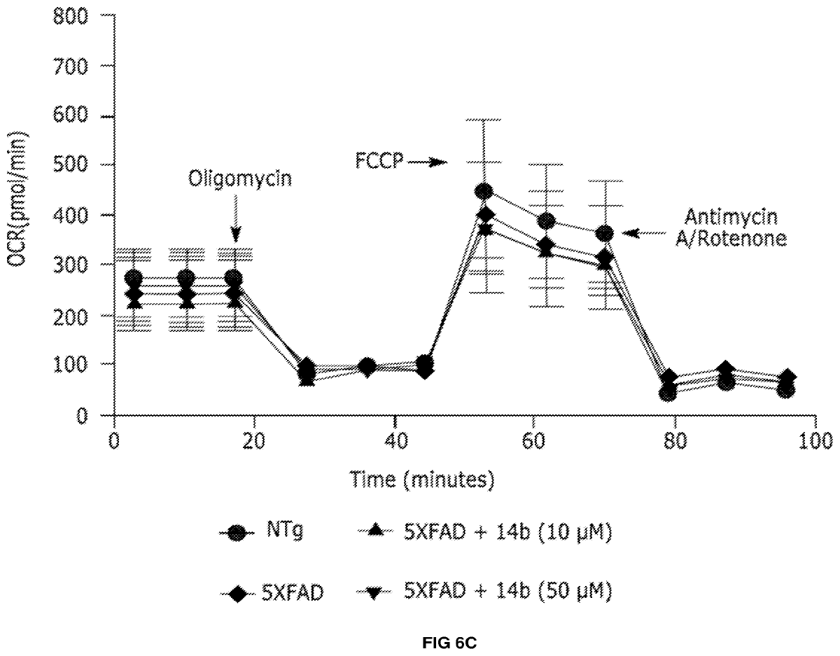

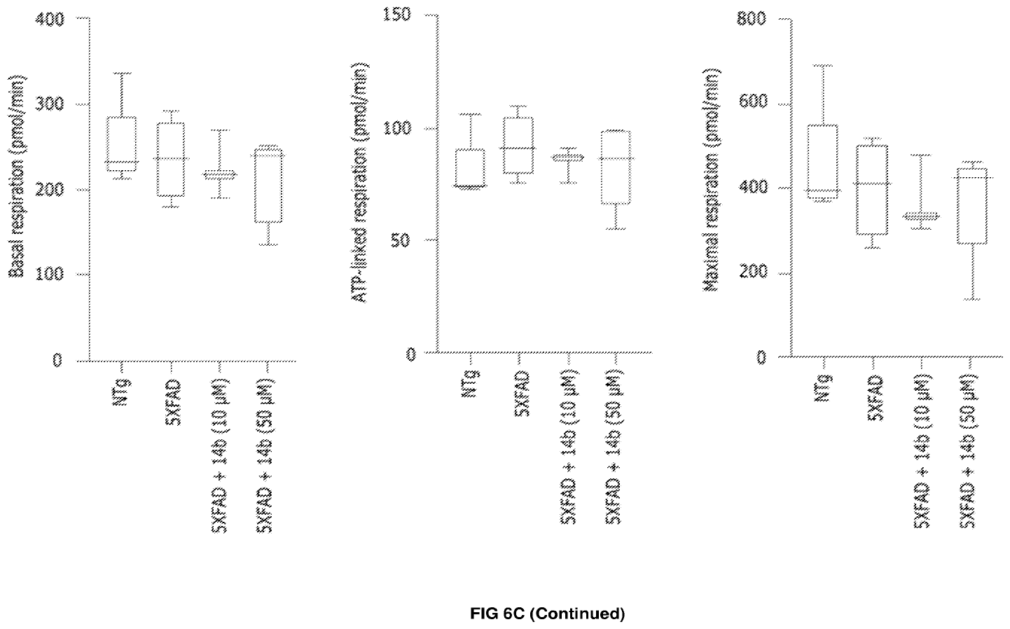

To obtain proof-of-concept protective effect of 14b in an ex vivo mouse model of AD we isolated primary mouse cortical neurons from the 5XFAD Alzheimer's model mouse (FIGS. 6A-6C). 5XFAD neurons were cultured for 10 days and then treated with 14b at 10 or 50 μM for 48 hours and then stained with the mitochondrial outer membrane protein TOM20 to evaluate mitochondrial morphological changes (FIG. 6A). Non-AD NTg mouse neurons were used as control. Both 10 and 50 μM concentration of 14b significantly rescued Aβ-induced defective mitochondrial morphology wherein both mitochondrial length and aspect ratio were significantly rescued compared with no treatment control in the 5XFAD mouse neurons (FIG. 6B). Similarly, to SH-SY5Y cells, compound 14b showed a trend to rescue mitochondrial dysfunction in the 5XFAD mouse neurons (FIG. 6C). Thus, compounds disclosed herein, e.g., compound 14b show promise to protect primary AD mouse model neurons.

### Example 25: Calculation of Multiparameter Optimization Scores

Multiparameter optimization (MPO) scores were calculated for compounds disclosed herein using a standard MPO equation, e.g., as disclosed in ACS Chem. Neurosci. 2016, 7, 6, 767-775. The MPO algorithm uses a weighted scoring function assessing the alignment of six key physicochemical properties (clogP, clogD, MW, TPSA, HBD, and pKa) generally considered indicative of druglike properties. The score is therefore useful for identifying candidates useful for central nervous system (CNS) therapy. CNS MPO scores range from 0 to 6.0, and higher scores are generally considered to be indicative of a greater potential for CNS therapeutic applications.

Table 3 below shows the multiparameter optimization scores for compounds disclosed herein.

TABLE 3

| Compound No. | MPO Score |
|---|---|
| 1 | 3.6 |
| 13a | 4.0 |
| 13b | 3.7 |
| 14b | 3.6 |
| 13c | 3.7 |
| 14c | 3.6 |
| 13d | 3.6 |
| 14d | 3.5 |
| 13? | 4.0 |
| 14e | 4.0 |
| 13f | 4.0 |
| 13g | 4.0 |
| 14g | 3.7 |
| 13h | 4.0 |
| 14h | 1.73 |
| 13i | 2.31 |
| 13j | 2.39 |
| 13k | 2.31 |
| 131 | 2.39 |

### REFERENCES

1. Hyman, B. T. et al., Alzheimers Dement. 2012, 8, 1-13.
2. Bateman, R. J. et al., N. Engl. J. Med. 2012, 367, 795-804.
3. Zetterberg, H. et al., J. Alzheimers Dis. 2013, 33 Suppl 1, S361-369.
4. Morsy, A. et al., J. Alzheimers Dis. 2019, 72, S145-S176.
5. Salloway, S. et al., N. Engl. J. Med. 2014, 370, 322-333.
6. Wirz, K. T. et al., J. Alzheimers Dis. 2014, 38, 719-740.
7. Reiman, E. M. et al., J. Alzheimers Dis. 2011, 26 Suppl 3, 321-329.
8. Grill, J. D. et al., Expert Rev. Neurother. 2010, 10, 711-728.
9. Blaikie, L. et al., Medchemcomm 2019, 10, 2052-2072.
10. Veitch, D. P. et al., Alzheimers Dement. 2019, 15, 106-152.
11. Toledo, J. B. et al.; Alzheimers Dement. 2017, 13, 965-984.
12. Moreira, P. I. et al., J. Alzheimers Dis. 2006, 9, 101-110.
13. Moreira, P. I. et al., Antioxid. Redox Signal. 2007, 9, 1621-1630.
14. Reddy, P. H. et al., Brain Res. Rev. 2005, 49, 618-632.
15. Yan, S. D. et al., Nature 1997, 389, 689-695.
16. Lustbader, J. W. et al., Science 2004, 304, 448-452.
17. He, X. et al., J. Steroid Biochem. Mol. Biol. 2003, 87, 191-198.
18. Ivell, R. et al., Endocrinology 2003, 144, 3130-3137.
19. Grimm, A. et al., Mol. Neurobiol. 2012, 46, 151-160.
20. Lim, Y. A. et al., PLOS One 2011, 6, e28887.
21. Morsy, A. et al., J. Med. Chem. 2019, 62, 4252-4264.
22. Yao, J. et al., Mol. Cell Neurosci. 2007, 35, 377-382.
23. Kissinger, C. R. et al., J. Mol. Biol. 2004, 342, 943-952.
24. Schmidt, M. et al., Int. J. Mol. Sci. 2020, 21.
25. Boutin, S. et al., Bioorg. Med. Chem. Lett. 2018, 28, 3554-3559.
26. Viswanath, A. N. I. et al., Chem. Biol. Drug Des. 2017, 90, 1041-1055.
27. Marques, A. T. et al., Int. J. Quantum Chem. 2008, 108, 1982-1991.
28. Xie, Y. et al., Bioorg. Med. Chem. Lett. 2006, 16, 4657-4660.
29. Valasani, K. R. et al., SChem. Biol. Drug Des. 2013, 81, 238-249.
30. Trippier, P. C., Curr. Med. Chem. 2016, 23, 1392-1407.
31. Ayan, D. et al., ChemMedChem 2012, 7, 1181-1184.
32. Zhang, H. Y., Acta. Pharmacol. Sin. 2012, 33, 1170-1175.
33. Xiao, X. et al., Neurobiol. Aging 2019, 81, 77-87.
34. Abreo, M. A. et al. U.S. Pat. No. 6,964,957 B2, 2005.
35. LaFerla, F. M. et al., Nat. Rev. Neurosci. 2007, 8, 499-509.
36. Manczak, M. et al., Hum. Mol. Genet. 2006, 15, 1437-1449.
37. Gouras, G. K. et al., Am. J. Pathol. 2000, 156, 15-20.
38. Rhein, V. et al., Cell Mol. Neurobiol. 2009, 29, 1063-1071.
39. Gray, N. E. et al., J. Alzheimers Dis. 2015, 45, 933-946.
40. Kuhla, B. et al., J. Neural Transm. (Vienna) 2004, 111, 427-439.
41. Ye, X. et al., Naunyn Schmiedebergs Arch. Pharmacol. 2014, 387, 75-85.
42. Kovalevich, J. et al., Methods Mol. Biol. 2013, 1078, 9-21.
43. Takahashi, R. H. et al., J. Neurosci. 2004, 24, 3592-3599.

What is claimed:

1. A compound, or pharmaceutically acceptable salt thereof, having the structure of Formula I: 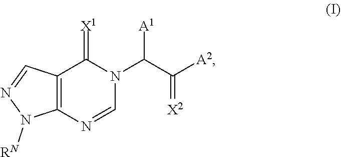
wherein
X¹ and X² are each independently O, S, or NR^{N};
each R^{N} is independently H or C₁₋₃ alkyl;
A¹ is C₃₋₁₂ cycloalkyl, 3-12 membered heterocycloalkyl having 1-4 ring heteroatoms selected from O, S, and N, C₆₋₁₀ aryl, or 5-12 membered heteroaryl having 1-4 ring heteroatoms selected from O, S, and N, wherein the cycloalkyl, heterocycloalkyl, aryl, or heteroaryl is optionally substituted with 1-3 R¹;
A² is 3-12 membered heterocycloalkyl having 1-4 ring heteroatoms selected from O, S, wherein the heterocycloalkyl is optionally substituted with 1-3 R²; and
each R¹ and R² is independently halogen, OH, CN, C₁₋₆ alkyl, C₁₋₆ alkyl substituted with 1-3 OH, C₁₋₆ alkyl substituted with 1-3 halogen, C₁₋₆ alkoxy, C₁₋₆ alkoxy substituted with 1-3 halogen, NO₂, NH₂, NH(C₁₋₆ alkyl), N(C₁₋₆ alkyl)₂, COOH, C(O)O—C₁₋₆ alkyl, C(O)NH₂, C(O)NH(C₁₋₆ alkyl), or C(O)N(C₁₋₆ alkyl)₂;
with the proviso that the compound is not 1-(azepan-1-yl)-2-phenyl-2-(4-thioxo-1,4-dihydro-5H-pyrazolo[3,4-d]pyrimidin-5-yl)ethan-1-one.

2. The compound or salt of claim 1, wherein at least one of X¹ and X² is O.

3. The compound or salt of claim 1 or 2, wherein X¹ is O.

4. The compound or salt of claim 1, wherein X¹ is S.

5. The compound or salt of any one of claims 1 to 4, wherein X² is O.

6. The compound or salt of any one of claims 1 to 5, wherein at least one R^{N} is H.

7. The compound or salt of any one of claims 1 to 6, wherein each R^{N} is H.

8. The compound or salt of any one of claims 1 to 7, wherein A¹ is C₆₋₁₀ aryl optionally substituted with 1-3 R¹.

9. The compound or salt of claim 8, wherein A¹ is phenyl optionally substituted with 1-3 R¹.

10. The compound or salt of claim 8 or 9, wherein A¹ is unsubstituted.

11. The compound or salt of claim 8 or 9, wherein A¹ is substituted with 1 R¹.

12. The compound or salt of claim 11, wherein R¹ is halogen.

13. The compound or salt of claim 12, wherein R¹ is F or Cl.

14. The compound or salt of claim 12 or 13, wherein R¹ is F.

15. The compound or salt of claim 12 or 13, wherein R¹ is Cl.

16. The compound or salt of any one of claims 1 to 15, wherein A² is 3-12 membered heterocycloalkyl having at least 1 ring N heteroatom.

17. The compound or salt of claim 16, wherein A² is 4-6 membered heterocycloalkyl having at least 1 ring N heteroatom.

18. The compound or salt of claim 17, wherein A² is 6 membered heterocycloalkyl having at least 1 ring N heteroatom.

19. The compound or salt of claim 18, wherein A² is morpholino or thiomorpholino.

20. The compound or salt of claim 16 or 17, having the structure of Formula (II): 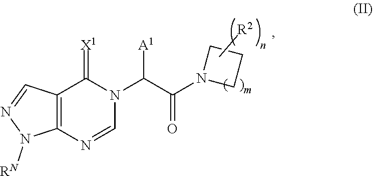
wherein
m is an integer from 1 to 3; and
n is an integer from 0 to 2.

21. The compound or salt of claim 20, wherein m is 1.

22. The compound or salt of claim 20, wherein m is 2.

23. The compound or salt of claim 20, wherein m is 3.

24. The compound or salt of any one of claims 20 to 34, wherein n is 0.

25. The compound or salt of any one of claims 20 to 23, wherein n is 1.

26. The compound or salt of any one of claims 20 to 23, wherein n is 2.

27. The compound or salt of any one of claim 1 to 23, 25, or 26, wherein R² is OH or C₁₋₆ alkoxy.

28. The compound or salt of claim 27, wherein R² is OH.

29. The compound or salt of claim 27, wherein R² is C₁₋₆ alkoxy.

30. The compound or salt of claim 29, wherein R² is OCH₃.

31. A compound, as recited in Table 1, or a pharmaceutically acceptable salt thereof.

32. The compound of claim 31 which is Compound 14b, 14h, or a pharmaceutically acceptable salt thereof.

33. A pharmaceutical composition comprising the compound or salt of any one of claims 1 to 32 and a pharmaceutically acceptable carrier or excipient.

34. The pharmaceutical composition of claim 33, further comprising one or more additional therapeutics.

35. The pharmaceutical composition of claim 34, wherein the one or more additional therapeutics comprise Aducanumab.

36. The compound of any one of claims 1 to 32 or the pharmaceutical composition of any one of claims 33 to 35 for use in inhibiting beta amyloid (Aβ)-amyloid-binding alcohol dehydrogenase (ABAD) protein-protein interaction in a subject in need thereof.

37. The compound or composition for use of claim 36, wherein inhibiting beta amyloid (Aβ)-amyloid-binding alcohol dehydrogenase (ABAD) protein-protein interaction comprises inhibiting beta amyloid (Aβ)-amyloid-binding alcohol dehydrogenase (ABAD) binding in a subject in need thereof.

38. The compound of any one of claims 1 to 32 or the pharmaceutical composition of any one of claims 33 to 35 for use in treating a disease or disorder capable of being modulated by inhibiting beta amyloid (Aβ)-amyloid-binding alcohol dehydrogenase (ABAD) protein-protein interaction in a subject in need thereof.

39. The compound or composition for use of claim 38, wherein inhibiting beta amyloid (Aβ)-amyloid-binding alcohol dehydrogenase (ABAD) protein-protein interaction comprises inhibiting beta amyloid (Aβ)-amyloid-binding alcohol dehydrogenase (ABAD) binding in a subject in need thereof.

40. The compound or composition for use of claim 38 or 39, wherein the disease or disorder is Alzheimer's disease, Parkinson's disease, motor neuron disease, or spinal muscular atrophy.

41. The compound or composition for use of claim 40, wherein the disease or disorder is Alzheimer's disease.

42. The compound or composition for use of any one of claims 36 to 41, further formulated for use with one or more additional therapeutics.

43. The compound or composition for use of claim 42, wherein the one or more additional therapeutics comprise Aducanumab.

44. Use of the compound of any one of claims 1 to 32 or the pharmaceutical composition of any one of claims 33 to 35 in the manufacture of a medicament for treating a disease or disorder capable of being modulated by inhibiting beta amyloid (Aβ)-amyloid-binding alcohol dehydrogenase (ABAD) protein-protein interaction in a subject in need thereof.

45. The use of claim 44, wherein the disease or disorder is Alzheimer's disease, Parkinson's disease, motor neuron disease, or spinal muscular atrophy.

46. The use of claim 45, wherein the disease or disorder is Alzheimer's disease.

47. The use of any one of claims 44 to 46, wherein the medicament further comprises one or more additional therapeutics.

48. The use of claim 47, wherein the one or more additional therapeutics comprise Aducanumab.

49. A method of inhibiting beta amyloid (Aβ)-amyloid-binding alcohol dehydrogenase (ABAD) protein-protein interaction comprising administering to a subject in need thereof a therapeutically effective amount of the compound or salt of any one of claims 1 to 32 or the pharmaceutical composition of any one of claims 33 to 35.

50. The method of claim 47, wherein inhibiting beta amyloid (Aβ)-amyloid-binding alcohol dehydrogenase (ABAD) protein-protein interaction comprises inhibiting beta amyloid (Aβ)-amyloid-binding alcohol dehydrogenase (ABAD) binding.

51. A method of treating or preventing a disease or disorder capable of being modulated by inhibiting beta amyloid (Aβ)-amyloid-binding alcohol dehydrogenase (ABAD) protein-protein interaction, comprising administering to a subject in need thereof a therapeutically effective amount of the compound or salt of any one of claims 1 to 32 or the pharmaceutical composition of any one of claims 33 to 35.

52. The method of claim 47, wherein inhibiting beta amyloid (Aβ)-amyloid-binding alcohol dehydrogenase (ABAD) protein-protein interaction comprises inhibiting beta amyloid (Aβ)-amyloid-binding alcohol dehydrogenase (ABAD) binding.

53. The method of claim 47 or 48, wherein the disease or disorder is Alzheimer's disease, Parkinson's disease, motor neuron disease, or spinal muscular atrophy.

54. The method of claim 49, wherein the disease or disorder is Alzheimer's disease.

55. The method of any one of claims 49 to 54, further comprising administering one or more additional therapeutics to the subject.

56. The method of claim 55, wherein the one or more additional therapeutics comprise Aducanumab.
